(19) United States
(12) Patent Application Publication
(10) Pub. No.: US 2023/0255931 A1
(43) Pub. Date: Aug. 17, 2023

(54) PHARMACEUTICAL COMPOSITIONS USEFUL FOR THE TREATMENT OF TISSUE INJURY

(71) Applicant: MedRegen, LLC, Baltimore, MD (US)

(72) Inventor: Zhaoli Sun, Baltimore, MD (US)

(21) Appl. No.: 18/189,741

(22) Filed: Mar. 24, 2023

Related U.S. Application Data
(63) Continuation of application No. 15/744,502, filed on Jan. 12, 2018, now abandoned.
(60) Provisional application No. 62/193,138, filed on Jul. 16, 2015.

(51) Int. Cl. A61K 31/395; A61K 9/00; A61K 9/08; A61K 31/506; A61K 31/4427; A61K 45/06; A61K 47/44; A61K 47/10; A61K 31/436; A61P 17/02; A61P 1/00; A61P 29/00

(52) U.S. Cl. CPC A61K 31/395; A61K 9/08; A61K 9/0019; A61K 31/436; A61K 31/506; A61K 31/4427; A61K 45/06; A61K 47/10; A61K 47/44; A61P 1/00; A61P 17/02; A61P 29/00

(57) ABSTRACT

The present invention relates to the field of tissue injury. More specifically, the present invention provides pharmaceutical compositions useful for the treatment of tissue injury. In certain embodiments, a pharmaceutical composition comprises (a) a stem cell mobilizer and (b) an immunosuppressive agent or non-immunosuppressive FK binding protein ligand. The pharmaceutical compositions of the invention can be administered by a variety of routes and dosing regimens.

### FIELD OF THE INVENTION

The present invention relates to the field of tissue injury. More specifically, the present invention provides pharmaceutical compositions useful for the treatment of tissue injury.

### BACKGROUND OF THE INVENTION

Stem cell therapy can be useful in the treatment of numerous conditions, including the treatment of organ transplantation, a variety of tissue injuries (wounds including bums, surgeries, bed sores, skin ulcers and the like), nerve injury and/or degeneration (including spinal cord injury), or the diagnosis of IBD or other inflammatory or autoimmune disease or the occurrence of an episode of IBD or other inflammatory or autoimmune disease. However, such therapy usually requires the isolation of endogenous stem cells from a subject, culturing and preparation of those stem cells outside the body, and then reimplantation into the subject. These processes are costly, time-consuming and are not always reliable.

Prior studies have shown that the administration of a stem cell mobilizer and an immunosuppressive agent at different times, or simultaneously but separately at different sites, can mobilize stem cells (see, e.g., Okabayashi T, et al. Mobilization of host stem cells enables long-term liver transplant acceptance in a strongly rejecting rat strain combination. Am J Transplant 2011; 11: 2046-2056 and Lin Q, Wesson RN, Maeda H, et al. Pharmacological mobilization of endogenous stem cells significantly promotes skin regeneration after full-thickness excision: The synergistic activity of AMD3100 and Tacrolimus. J Invest Dermatol 2014; 134(9): 2458-2468). Although the mobilization of stem cells under these regimes was better than that seen with administering either a stem cell mobilizer and an immunosuppressive agent alone, each still required multiple and multi-site injections of drugs, with an increased discomfort and risk to the subject and a decreased efficiency to the treatment regimen.

Thus there exists a great need for pharmaceutical formulations useful for mobilizing endogenous stem cells from their reservoirs in the bone marrow so that they accumulate in the area of tissue injury and aid in healing, without first having to remove and reimplant the stem cells, and which can be administered more efficiently to a subject with less risk and discomfort.

### SUMMARY OF THE INVENTION

In one embodiment, the present invention provides pharmaceutical compositions. In certain embodiments, the pharmaceutical composition comprises (a) a stem cell mobilizer; (b) an immunosuppressive agent; and optionally (c) a pharmaceutically acceptable carrier. In other embodiments, the composition provides (a) at least one stem cell mobilizer (b) at least one immunosuppressive agent; and optionally (c) a pharmaceutically acceptable carrier. In one embodiment, the pharmaceutical composition is formulated for administration substantially simultaneously at a single site on a subject, for example in a single formulation.

In another embodiment, the stem cell mobilizer comprises a CXCR4 antagonist. For example, the CXCR4 antagonist can comprise AMD3100, TG-0054, or AMD3465. In another embodiment, the CXCR4 antagonist comprises AMD3100. In another embodiment, the immunosuppressive agent comprises an FK binding protein ligand., for example Tacrolimus (FK-506) or an analog thereof. Examples of a Tacrolimus analogs include ascomycin, 506BD and L-685,818. In particular embodiments, the immunosuppressive agent comprises an immunosuppressor, for example Tacrolimus and wherein the at least one stem cell mobilizer comprises AMD3100. In certain embodiments, the Tacrolimus and AMD3100 are present in a ratio of about ⅒ to about 1/100.

The present invention also provides a pharmaceutical composition comprising (a) Tacrolimus; (b) AMD3100; and optionally (c) a pharmaceutically acceptable carrier, wherein the Tacrolimus and AMD3100 are present in a ratio of about ⅒ to about 1/100. In certain embodiments, the pharmaceutical composition is formulated for subcutaneous injection. In other embodiments, a pharmaceutical composition consists essentially of (a) Tacrolimus; and (b) AMD3100, wherein the Tacrolimus and AMD3100 are present in a ratio of about ⅒ to about 1/100.

The present invention further provides a pharmaceutical composition comprising a (a) CXCR4 antagonist and (b) an FK binding protein ligand. The FK binding protein ligand can comprise, for example, Tacrolimus or an analog thereof, meridamycin or synthetic ligand of FKBP (SLF). In other embodiments, the CXCR4 antagonist comprises AMD3100, TG-0054, or AMD3465.

In other embodiments, the pharmaceutical composition comprises (a) a stem cell mobilizer; (b) an immunosuppressive agent or non-immunosuppressive FK binding protein ligand; and optionally (c) a pharmaceutically acceptable carrier. The pharmaceutical composition can comprise (a) a stem cell mobilizer; (b) Tacrolimus; and optionally (c) a pharmaceutically acceptable carrier. In another embodiment, the pharmaceutical composition can comprise (a) one of AMD3100, TG-0054 or AMD3465; (b) an immunosuppressive agent or non-immunosuppressive FK binding protein ligand; and optionally (c) a pharmaceutically acceptable carrier. In one embodiment, the pharmaceutical composition is formulated for administration substantially simultaneously at a single site on a subject.

This invention also features pharmaceutical packs or kits containing one or more stem cell mobilizing agents and one or more immunosuppressants or nonimmunosuppresant FK binding protein ligands.

The pharmaceutical compositions and pharmaceutical packs or kits of the present invention may feature higher order combinations of stem cell mobilizing agents and immunosuppressants or nonimmunosuppresant FKBP ligands, for example one, two or more stem cell mobilizing agents (AMD3100, G-CSF etc.) may be combined with one, two or more immunosuppressants (e.g., FK506, Rapamycin, Cyclosporine A) or FKBP ligands (e.g., meridamycin).

In another embodiment, the present invention provides methods for treating tissue injury in a patient comprising the step of administering a therapeutically effective pharmaceutical composition of the invention. In certain embodiments, the administering step comprises substantially simultaneous administration of the stem cell mobilizer and immunosuppressant agent or nonimmunosuppresant FKBP ligands which comprise the pharmaceutical composition at a single site on the subject, for example as a single formulation. Tissue injury that can be treated with the pharmaceutical compositions and methods of the invention can comprise wounds, inflammatory or autoimmune diseaes such as inflammatory bowel disease, damage to or degeneration of peripheral nerves, for example from spinal cord injury (including acute injury and delayed secondary degeneration) or diabetic neuropathy, and organ transplantation. Wounds can include cutaneous wounds, for example, lacerations, bums, bed sores, and chronic wounds such as pressure ulcers or diabetic foot ulcers, or other wounds associated with diabetes.

The invention further provides a triple dosing regimen for a subject suffering from a tissue injury, or a method of treating a tissue injury in a subject, comprising administering a pharmaceutically effective amount of a pharmaceutical composition of the invention to the subject at about one month, about two months and about three months after a tissue injury, for a total of three administrations. In some embodiments, the administrations are subcutaneous or intramuscular. In one embodiment of the triple dosing regimen, the administering step comprises substantially simultaneous administration of the stem cell mobilizer and immunosuppressant agent or nonimmunosuppresant FKBP ligands which comprise the pharmaceutical composition at a single site on the subject, for example as a single formulation. In other embodiments, the tissue injury is selected from the group consisting of organ transplant, a burn, a wound, nerve injury and/or degeneration (including spinal cord injury), the diagnosis of IBD or other autoimmune or inflammatory disease and the occurrence of an episode of IBD or other autoimmune or inflammatory disease.

The invention further comprises a dosing regimen for a subject suffering from a tissue injury, or a method of treating a tissue injury in a subject, comprising administering a pharmaceutically effective amount of a pharmaceutical composition of the invention to the subject every other day for a predetermined time or until the tissue injury is healed or no longer observed. In some embodiments, the administrations are subcutaneous or intramuscular. In other embodiments, the administering step comprises substantially simultaneous administration of the stem cell mobilizer and immunosuppressant agent or nonimmunosuppresant FKBP ligands which comprise the pharmaceutical composition at a single site on the subject, for example as a single formulation. In other embodiments, the tissue injury is selected from the group consisting of organ transplant, a burn, a wound, nerve injury and/or degeneration (including spinal cord injury), the diagnosis of IBD or other autoimmune or inflammatory disease and the occurrence of an episode of IBD or other autoimmune or inflammatory disease.

### BRIEF DESCRIPTION OF THE FIGURES

FIG. 1 is a graph showing stem cell mobilization activity by the AMD3100/Tacrolimus (FK506) AF Combination formulation.

FIG. 2 is a photographic array showing AF Combination treatment in a mouse model of bums.

FIG. 3 is a graph showing AF Combination treatment accelerated wound healing in a mouse model of burns.

FIG. 4A is a photographic array and FIG. 4B is a graph showing AF Combination treatment ameliorated scar formation at 5 months post-burns.

FIG. 5 is a graph showing stem cell mobilization activity by an AF Combination compared to saline, AMD3100 alone, Tacrolimus alone and separate injections of AMD3100 and Tacrolimus, with an n=3 per group.

FIG. 6A is a diagram showing the production of wounds in diabetic rats, with photographs of the representative wounds at days 0 and 9.

FIG. 6B is a diagram and graph showing measurement of colony forming cells (CFC) in peripheral blood samples at 3 hours after saline or AF Combination treatment in diabetic rats.

FIG. 6C is a panel of photographs showing wound healing in diabetic rats after treatment with either saline or an AF Combination.

FIG. 6D is a panel of photographs of saline and AF Combination treated diabetic rats showing the number of CD133+ endothelial progenitor cells (“CD133”); CD34+ stem cells (“CD34”); capillary and hair follicle neogenesis (“Endothelium”) and scarring (“Scars at 3 months”) at the wound sites of saline or AF Combination treated diabetic rats.

FIG. 7A is a series of photographs showing the creation of incisional wounds (4 cm) in aged mice.

FIG. 7B is a diagram and photographs showing the measurement of healing wound tensile strength in aged mice.

FIG. 7C are graphs showing the tension at break (in Newtons) and the work at break (in millijoules) for healing wound in aged mice treated with saline, a stem cell mobilizer (“AMD”), an immunosuppressive agent (“FK506”), and AF Combination (“AF”).

FIG. 8A is an array of photographs of gel electrophoreses indicating expression of SDF-1, CD34, CD133 and Ki67 in the wound sites of aged mice treated with saline, a stem cell mobilizer (“AMD”), an immunosuppressive agent (“FK”), and an AF Combination (“AF”).

FIG. 8B is an array of photographs showing epithelial proliferation (Ki67+) at the wound sites 7 days after wounding in aged mice treated with saline, a stem cell mobilizer (“AMD”), an immunosuppressive agent (“FK”), and an AF Combination (“AF Combo”).

FIG. 8C is an array of photographs showing scar formation in aged mice treated with saline, a stem cell mobilizer (“AMD3100”), an immunosuppressive agent (“FK506”), and an AF Combination (“AF Combo”), stained with either hematoxylin and eosin (“H&E”) or Masson’s trichrome.

FIG. 9A is an array of photographs showing bloody and loose stools observed in mice with DSS-induced IBS treated with saline as compared with those treated with an AF combination (“AF”) at day 10 post-DSS administration.

FIG. 9B is a photograph and a graph showing a shorter colon and enlarged cecum observed in mice with DSS-induced IBS treated with saline as compared with those treated with an AF combination (“AF”) at day 10 post-DSS administration (in the graph, * p<0.05; n=4).

FIG. 9C is a set of photographs showing hematoxylin and eosin (“H&E”) stained histological sections of colon showing the loss, disruption, or shortening of the crypts and apparent infiltration of inflammatory cells in lamina propria/mucosa observed in mice with DSS-induced IBS treated with saline (“control”) as compared with those treated with an AF combination (“AF”) at day 10 post-DSS administration.

FIG. 10 is a graph showing the disease activity index (DAI) mice with DSS-induced IBS treated with saline or an AF Combination (“AF”).

FIG. 11 is set of photographs showing immunofluorescence double staining of CD133+ stem cells and Ki67+ proliferating cells in colonic mucosa from mice with DSS-induced IBS treated with saline or an AF Combination (“AF”).

FIG. 12A is a set of photographs showing mucosal inflammation and rectal prolapse in a representative mouse that had developed spontaneous colitis (“IL-10-/- mice”) at day 0 (left-hand panel) and day 28 (right-hand panel) after treatment with an AF Combination.

FIG. 12B is a set of photographs showing a portion of the colon with feces from mice that had developed spontaneous colitis (“IL-10-/-”), treated with either saline (“control”) or an AF Combination (“AF”).

FIG. 12C is a set of photographs showing hematoxylin and eosin (“H&E”) stained histological sections of colon from wild-type mouse or mice that had developed spontaneous colitis (“IL-10-/-”) treated with either saline (“saline control”) or an AF Combination (“AF”). The lower panels show the inset indicated in the upper panels at a higher magnification.

FIG. 13 is a graph representing the behavioral assessment four weeks following moderate contusive spinal cord injury in rats. Treatment with an AF Combination was begun at day 1 or day 5 following injury. The BBB score for the left and right limb movement was evaluated for 30 days following injury. Values represent mean +/- SEM of both left and right limbs, and statistical significance (p<0.05) relative to saline control group is denoted by asterisks.

FIG. 14 is set of photographs showing hematoxylin and eosin (“H&E”) staining of spinal cords from rats subjected to moderate contusive spinal cord injury and treated with either an AF Combination (panel A; “AF treatment”) or saline (panel B; “Saline control”), and a graph (panel C) representing the size of the cavity at the injury site in mm2 for saline control rats (“Saline”) or rats treated with an AF Combination (“AF combo”). In the graph, the values represent area measurement across groups subjected to Tukey post hoc tests, and statistical significance (p<0.05) relative to the saline control group is denoted by an asterisk.

FIG. 15 is set of photographs showing skin regeneration from day 0 through 12 weeks, in surgical excisional wounds in swine that were transplanted with skin allografts. The lower left-hand panel shows the regeneration of hair follicles in the newly-grown skin at 12 weeks, and arrows show histologic staining (hematoxylin and eosin) of the indicated areas (right-hand panels).

FIG. 16 is a graph showing the improved rate of wound closure in injured rats treated with an AF Combination (“AF combo”) or separate injections of AMD3100 and Tacrolimus (A+F) as a function of percent original wound area over time in days post-injury, to complete wound closure.

### DETAILED DESCRIPTION OF THE INVENTION

It is understood that the present invention is not limited to the particular methods and components, etc., described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to a “protein” is a reference to one or more proteins, and includes equivalents thereof known to those skilled in the art and so forth. Specific methods, devices, and materials are described, although any methods and materials similar or equivalent to those described herein can be used in the practice or testing of the present invention.

All publications cited herein are hereby incorporated by reference including all journal articles, books, manuals, published patent applications, and issued patents. In addition, the meaning of certain terms and phrases employed in the specification, examples, and appended claims are provided. The definitions are not meant to be limiting in nature and serve to provide a clearer understanding of certain aspects of the present invention.

“Agent” or “active ingredient” refers to any materials that may be used as or in pharmaceutical compositions that can generate a pharmaceutical effect, for example compounds such as small synthetic or naturally derived organic compounds, nucleic acids, polypeptides, antibodies, fragments, isoforms, variants, or other materials that may be used independently for such purposes, all in accordance with the present invention.

“Antagonist” refers to an agent that suppresses or inhibits at least one bioactivity, for example of a protein, cell or physiologic system. An antagonist can be a compound which inhibits or decreases the interaction between a protein or cellular receptor and another molecule, e.g., a target peptide or enzyme substrate. An antagonist may also be a compound that down-regulates expression of a gene or which reduces the amount of expressed protein related to the bioactivity to be antagonized.

“Hematopoiesis” refers to the highly orchestrated process of blood cell development and homeostasis. Prenatally, hematopoiesis occurs in the yolk sack, then liver, and eventually the bone marrow. In normal adults it occurs in bone marrow and lymphatic tissues. All blood cells develop from pluripotent stem cells. Pluripotent cells differentiate into stem cells that are committed to three, two or one hematopoietic differentiation pathways.

The term “immunosuppressive agent” is used interchangeably with “immunosuppressant agent” and refers to an agent that inhibits, slows or reverses the activity of the immune system. Immunosuppressive agents act, for example, by suppressing the function of responding immune cells (including, for example, T cells), directly (e.g., by acting on the immune cell) or indirectly (by acting on other immune-mediating cells).

The terms “stem cells” and “hematopoietic stem cells” are used interchangeably herein. Stem cells can be distinguished from other cell types by two important characteristics. First, stem cells are unspecialized cells capable of renewing themselves through cell division, sometimes after long periods of inactivity. Second, under certain physiologic or experimental conditions, stem cells can be induced to become tissue- or organ-specific cells with special functions. In some organs, such as the gut and bone marrow, stem cells regularly divide to repair and replace worn out or damaged tissues. In other organs, however, such as the pancreas and the heart, stem cells only divide and differentiate under special conditions. As used herein, the term “stem cells” can refer to multipotent or pluripotent stem cells that are capable of differentiating into all blood cells including erythrocytes, leukocytes and platelets. For instance, the “hematopoietic stem cells” or “stem cells” as used in the invention are contained not only in bone marrow but also in umbilical cord blood derived cells.

The term “endogenous stem cells” means stem cells derived from the same individual which is being treated. As used herein, “endogenous stem cells” can be removed from the subject and reimplanted, or can remain in the subject throughout the course of treatment. The term “autochthonous stem cells” means stem cells which are native to the subject being treated, and generally indicates that the stem cells remain in the subject the course of treatment. It is understood, when stem cell mobilizers are administered to a subject according to the methods described herein, that endogenous/autochthonous stem cells are mobilized.

A “stem cell mobilizer,” “mobilizer of hematopoietic stem cells or progenitor cells” or “mobilize” used with respect to stem cells refers to any compound, for example a small organic molecule, synthetic or naturally derived compound, a polypeptide or protein, such as a growth factor or colony stimulating factor or an active fragment or mimic thereof, a nucleic acid, a carbohydrate, an antibody, or any other agent that acts to enhance the migration of stem cells from the bone marrow into the peripheral blood. A stem cell mobilizer can increase the number of hematopoietic stem cells or hematopoietic progenitor/precursor cells in the peripheral blood, thus allowing for a more accessible source of stem cells for use in treating subjects according to the present methods, for example organ transplant recipients, bum victims, those with autoimmune or inflammatory diseases such as IBD, those nerve injury and/or degeneration (including spinal cord injury) or those in need of promoting wound healing, including wounds associated with diabetes. In some embodiments, a stem cell mobilizer refers to any agent that mobilizes CD34+ and/or CD133+ stem cells. In other embodiments, a stem cell mobilizer disrupts CXCL12 (SDF-1)-mediated chemoattraction of CXCR4-expressing cells.

The terms “patient,” “subject,” or “host” are used interchangeably herein, and refer to any individual human or animal to be treated by the present methods, for example a human or non-human primate, bovine, ovine, porcine, feline, canine or rodent.

As used herein, the terms “treatment,” “treating,” “treat” and the like, refer to obtaining a desired pharmacologic or physiologic effect. The pharmacologic and/or physiologic effect can be prophylactic, for example by completely or partially delaying or preventing a particular outcome relating to a disease or disorder, or a symptom thereof, or may be therapeutic, for example by ameliorating or causing a partial or complete cure for a disease or disorder /or symptom or adverse effect thereof.

The present invention provides pharmaceutical compositions comprising at least one stem cell mobilizer and at least one immunosuppressive agent or non-immunosuppressive FK binding protein ligand. Suitable stem cell mobilizers are known in the art, and include small organic molecules, polypeptides, nucleic acids, and carbohydrates.

Suitable polypeptide stem cell mobilizers can comprise a cytokine, a colony stimulating factor, a protease or a chemokine. In some embodiments, the cytokine stem cell mobilizers include interleukin-1 (IL-1), interleukin-3 (IL-3), interleukin-6 (IL-6), interleukin-11 (IL-11), interleukin-7 (IL-7), and interleukin-12 (IL12).

Suitable colony stimulating factor stem cell mobilizers can comprise granulocyte colony stimulating factor (G-CSF), granulocyte-macrophage colony stimulating factor (GM-CSF), macrophage colony stimulating factor (M¬CSF), stem cell factor, FLT-3 ligand or combinations thereof.

Suitable protease stem cell mobilizers can comprise metalloproteinase (like MMP2 or MMP9) a serine protease, (like cathepsin G, or elastase) a cysteine protease (like cathepsin K) and a dipeptidyl peptidase-1 (DDP-1 OR CD26).

Suitable chemokine stem cell mobilizers can comprise CXCL12, IL-8, Mip-ia, and Gro3.

Suitable nucleic acid stem cell mobilizers can comprise a DNA or an RNA molecule, for example a small interfering RNA (siRNA) molecule or an antisense molecule.

Suitable carbohydrate stem cell mobilizers can comprise a sulfated carbohydrate, for example Fucoidan or sulfated dextran. Fucoidan is a carbohydrate consisting of L-fucose, sulfate and acetate in a molar proportion of 1:1.23:0.36 and can be isolated from the Pacific brown seaweed Fucus evanescens. See Bilan et al., 337(8) Carbohydrate Research 719-30 (2002). Sulfated dextrans refer to a series of polysaccharides that have variable sulfated patterns and Pomin et al., 15(12) Glycobiology 1376-1385 (2005); Melo et al., 279(2) J. Biol. Chem. 20824-20835 (2004); and Farias et al., 275(38) J. Biol. Chem. 29299-29307 (2000), the entire disclosures of which are herein incorporated by reference.

Other suitable stem cell mobilizers include AMD3100; stromal cell-derived factor (SDF-1); SDF-1 analogs (e.g., CTCE-0214 available for example from Chemokine Therapeutics Corp.); anti-SDF-1 antibodies; cyclophosphamide; stem cell factor (SCF); filgrastim; ancestim; Myeloid Progenitor Inhibitory Factor-1 (MPIF-1), as disclosed in, e.g., U.S. Patent Publication No. 20080274109, the entire disclosure of which are herein incorporated by reference; and Very Late Antigen (VLA-4) antagonists such as an alpha-4 integrin antagonist like Natalizumab or Anti-phospho-Integrin ct4 (Ser988), clone 6.33 (Upstate Cell Signaling Solutions), or a peptide (e.g., phenylacetyl-leu-asp-phe-D-prolineamide available, for example, from Cytel Corp., San Diego Calif.).

In certain embodiments, the stem cell mobilizer comprises a CXCR4 antagonist. In some embodiments, the CXCR4 antagonist is TG-0054 (Burixafor; Phosphonic acid, p-(2-(4-(6-amino-2-(((trans-4-(((3-(cyclohexylamino)propyl)amino)methyl)-cyclohexyl)methyl)amino)-4-pyrimidinyl)-1-piperazinyl)ethyl)-) (TaiGen Biotechnology Co., Ltd. (Taipei, Taiwan)). In other embodiments, the CXCR4 antagonist is AMD3465 (N-(pyridin-2-ylmethyl)-1-[4-(1,4,8,11- tetrazacyclotetradec-1-ylmethyl)phenyl]methanamine). In yet other embodiments, the CXCR4 antagonist is AMD3100. AMD3100, also known as(1,1′-[1,4-phenylenebis(methylene)]bis¬1,4,8,11-tetraazacyclo-tetradecane, is a symmetric bicyclam, prototype non-peptide antagonist of the CXCR4 chemokine receptor described, for example, in U.S. Pats. No. 6,835,731 and No. 6,825,351, the entire disclosures of which are herein incorporated by reference. The term “AMD3100” is used interchangeably herein with Plerixafor, rINN, JM3100, and the trade name, Mozobil™. The present invention also contemplates using mimetics of AMD3100 in the present pharmaceutical compositions. For example, mutational substitutions at 16 positions located in TM-III, -IV, -V, -VI, and -VII lining the main ligand-binding pocket of the CXCR4 receptor have identified three amino acid residues as the main interaction points for AMD3100; namely Asp¹⁷¹ (AspIV:20), Asp²⁶² (AspVI:23), and Glu²⁸⁸ (GluVII:06). Molecular modeling suggests that one cyclam ring of AMD3100 interacts with Asp¹⁷¹ in TM-IV, whereas the other ring is sandwiched between the carboxylic acid groups of Asp²⁶² and Glu²⁸⁸ from TM-VI and -VII, respectively. In one study, it was found that introduction of only a Glu at position VII: 06 and the removal of a neutralizing Lys residue at position VII:02 resulted in a 1000-fold increase in affinity of AMD3100 to within 10-fold of its affinity in CXCR4. Any other suitable AMD3100 mimetic can be used, such as for example, peptide or non-peptide antagonists with improved oral bioavailability which are designed to efficiently and selectively block the CXCR4 receptor.

In other embodiments, the stem cell mobilizer is BKT140 (Biokin Therapeutics, Ltd. (Rehovot, Israel). BKT140, also known as 4F-benzoyl-TN14003, binds to and inhibits the CXCR4 chomokin receptor with high affinity, with an IC₅₀ of ~ 1 nmol/L compared with the values obtained with AMD3100. Moreover, BKT140 hinders the cell migration stimulated by CXCL12 within IC₅₀ values of 0.5 to 2.5 nmol/L, compared with IC50 value of 51 ± 17 nmol/L for Plerixafor, suggesting ahigh mobilization capacity. See, e.g., Peled et al., 20 Clin Cancer Res. 469-79 (2013), the entire disclosure of which is herein incorporated by reference.

As discussed above, the pharmaceutical compositions of the invention can comprise at least one immunosuppressive agent with the at least one stem cell mobilizer.

Any suitable immunosuppressive agent can be used in the present pharmaceutical compositions, including: a calcineurin inhibitor (e.g., cyclosporin (CsA) and analogs thereof, ISA(TX) 247, and Tacrolimus); azathioprine (AZ); mycophenolate mofetil (MMF); mizoribine (MZ); leflunomide (LEF); adrenocortical steroids (also known as adrenocortical hormones, corticosteroids, or corticoids) such as prednisolone and methylprednisolone; sirolimus (also known as rapamycin); everolimus; FK778; TAFA-93; deoxyspergualin (DSG); and 2-amino-2-[2-(4- octylphenyl)ethyl]-1,3-propanediol hydrochloride (FTY720).

Other suitable immunosuppressive agents include: cyclophosphamide; 15-deoxyspergualin (Gusperimus); interferons; sulfasalazine; mimoribine; misoprostol; anti-IL-2 receptor antibodies; thalidomide; anti-tumor necrosis factor antibodies; anti-CD2 antibodies; anti-CD147 antibodies; anti-CD4 antibodies; anti-CD8 antibodies and anti-thymocyte globulin antibodies; ORTHOCLONE® (also known as OKT3, from Ortho Biotech, Raritan, N.J.); SANDIMMUNE® ORAL (cyclosporine), available for example from Sandoz Pharmaceuticals, Hanover, N.J.; PROGRAF®, also known as Tacrolimus, available for example from Fujisawa Pharmaceuticals, Deerfield, Ill.); CELLCEPT®, also known as mycophenolate, available for example from Roche Pharmaceuticals, Nutley, N.J.; and RAPAMUNE®, also known as sirolimus, available for example from Pfizer, Inc, Collegeville, Pa.). In some embodiments, the immunosuppressive agent is rapamycin, Tacrolimus, mycophenolic acid, azathioprine or cyclophosphamide. Still other suitable immunosuppressive agents include an interleukin-2 alpha-chain blocker (e.g., basiliximab and daclizumab); an inhibitor of inosine monophosphate dehydrogenase (e.g., mycophenolate mofetil); or an inhibitor of dihydrofolic acid reductase (e.g., methotrexate).

In certain embodiments, the immunosuppressive agent is Tacrolimus. Tacrolimus (also known as FK-506 or Fujimycin) is an immunosuppressive drug that is mainly used after allogeneic organ transplant to reduce the activity of the patient’s immune system, and so lower the risk of organ rejection. It reduces interleukin-2 (IL-2) production by T-cells. Tacrolimus is also used in a topical preparation for the treatment of severe atopic dermatitis (eczema), severe refractory uveitis after bone marrow transplants, and the skin condition vitiligo. Tacrolimus is a 23- membered macrolide lactone discovered in 1984 from the fermentation broth of a Japanese soil sample that contained the bacteria Streptomyces tsukubaensis. The drug is sold under the trade names Prograf® given twice daily (intravenous); Advagraf®, which is a sustained release formulation allowing once daily dosing (oral); and Protopic®, which is a topical formulation.

The pharmaceutical compositions of the invention can also comprise at least one FK binding protein ligand with at least one stem cell mobilizer. Examples include FK-506 (Tacrolimus) and derivatives/analogs thereof, including 506BD and L0685,818; rapamycin and derivatives/analogs thereof including Way-124466, RAD001, CCI-779, and AP23573; ascomycin and derivatives/analogs thereof including pimecrolimus. See, e.g., Liu et al., 23(11) EXPERT OPIN. THER. PATENTS 1435-49 (2013), the entire disclosure of which is herein incorporated by reference. Furthermore, although the immunosuppressive agent Tacrolimus/FK-506 is an FK binding protein ligand, in certain embodiments, an FK binding protein ligand can comprise a non-immunosuppressive FK binding protein ligand. Examples of non-immunosuppressive ligands include meridamycin, antascomicins, and synthetic ligand of FKBP (SLF).

Accordingly, the present invention provides a pharmaceutical composition comprising an effective amount of at least one stem cell mobilizer and at least one immunosuppressive agent or non-immunosuppressive FK binding protein ligand. In certain embodiments, the present invention further contemplates a pharmaceutical composition comprising a single active agent that has characteristics of both a stem cell mobilizer and an immunosuppressive agent or non-immunosuppressive FK binding protein ligand. For example, Tacrolimus can be used as both a stem cell mobilizer and an immunosuppressive agent.

As used herein, an “effective amount” or a “therapeutically effective amount” is used interchangeably and refers to an amount of a pharmaceutical composition of the present invention which provides the desired treatment of a subject. As would be appreciated by one of ordinary skill in the art, the therapeutically effective amount of the present pharmaceutical compositions to treat a given disease, disorder or condition will vary from subject to subject, depending on factors such as age, general condition of the subject, the severity of the condition being treated, the particular compound and/or composition administered, and the like. An appropriate therapeutically effective amount of the present pharmaceutical compositions suitable for any individual subject can be readily determined by one of ordinary skill in the art from the information provided herein.

The pharmaceutical compositions of the present invention are in biologically compatible form suitable for administration to subjects, for example to humans. The pharmaceutical compositions can further comprise a pharmaceutically acceptable excipient. The term “pharmaceutically acceptable” means suitable for use in humans or animals, for example as approved by a governmental regulatory agency (such as the US Food and Drug Administration) or listed in the U.S. Pharmacopeia or other generally recognized pharmacopeia, or which are generally recognized as safe (GRAS).

As used herein, the term “excipient” refers to a carrier or vehicle (including any suitable diluent, adjuvant or the like) with which the stem cell mobilizer and/or the immunosuppressive agent are administered. Suitable pharmaceutically acceptable excipients can be sterile liquids, such as water and oils, including those of petroleum, animal, vegetable or synthetic origin such as peanut oil, soybean oil, mineral oil, sesame oil and the like. Water can be a pharmaceutically acceptable excipient when the pharmaceutical composition is administered orally. Sterilized water, saline, aqueous dextrose, glycerol, lactated Ringer’s solution and the like can be pharmaceutically acceptable excipients when the pharmaceutical composition is injected, such as administered subcutaneously, intramuscularly, or intravascularly (for example intravenously).

Other suitable pharmaceutically acceptable excipients include starch, glucose, lactose, sucrose, gelatin, malt, rice, flour, chalk, silica gel, sodium stearate, glycerol monostearate, talc, sodium chloride, dried slim milk, glycerol, propylene, glycol, water, ethanol and the like. The pharmaceutical composition can also contain minor amounts of wetting or emulsifying agents, or pH buffering agents.

The pharmaceutical compositions of the present invention can take any suitable form for administration to a subject, such as a human subject, for example solutions, suspensions, emulsions, tablets, pills, capsules, powders, sustained-release formulations and the like. The present pharmaceutical composition can also, for example, be formulated as a suppository, with traditional pharmaceutical excipients such as triglycerides. Oral pharmaceutical formulations of the invention can include standard carriers as pharmaceutical excipients, such as pharmaceutical grades of mannitol, lactose, starch, magnesium stearate, sodium saccharine, cellulose, magnesium carbonate, etc. In a specific embodiment, a pharmaceutical composition of the invention comprises an effective amount of a stem cell mobilizer and/or an immunosuppressive agent together with a suitable amount of a pharmaceutically acceptable excipient so as to provide the form for proper administration to the patient, for example by subcutaneous, intramuscular, or intravascular (for example intravenous) administration. For a discussion of the properties of solid and liquid pharmaceutically acceptable excipients which are suitable for use in the present pharmaceutical formulations, see, e.g., the excipients described in the Rowe et al., eds., Handbook of Pharmaceutical Excipients, 7th Edition, London: Pharmaceutical Press, 2012, which is incorporated herein by reference.

The pharmaceutical compositions of the present invention can be administered by any suitable route of administration, for example oral, parenteral, subcutaneous, intramuscular, intravenous, intra-arterial, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intraosseous, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, iontophoretic means, or transdermal means. In certain embodiments, the routes of administration for the present pharmaceutical compositions are oral administration or by injection, for example by subcutaneous, intramuscular, or intravascular (for example intravenous or intra-arterial) injection. In certain embodiments, the route of administration for the present pharmaceutical compositions is by subcutaneous injection.

In some embodiments, the pharmaceutical compositions of the invention comprising a stem cell mobilizer and an immunosuppressive agent can be used alone, e.g., a formulation comprising a stem cell mobilizer and an immunosuppressive agent without any other active ingredient, or in concert with at least one other active ingredient at appropriate dosages of the at least one other active ingredient as are known in the art to achieve a desired treatment, for example as defined by routine testing in order to obtain optimal efficacy while minimizing any potential toxicity.

Suitable therapeutically effective amounts and dosage regimens utilizing a pharmaceutical composition of the invention can be selected by the ordinarily skilled clinician in accordance with a variety of factors, including species, age, weight, sex, and overall medical condition of the patient; the condition to be treated and its severity or penetration; the route of administration; the renal and hepatic function of the patient; and the particular pharmaceutical composition employed.

In certain embodiments, the immunosuppressive agent comprising pharmaceutical compositions of the invention can be administered in low dose amount. The phrase “low dose” or “low dose amount” of an immunosuppressive agent in the context of the present invention (in combination with a stem cell mobilizer) refers to the use of a particular amount of an immunosuppressive drug that is lower than typically used for immunosuppression, for example lower than typically used for immunosuppression in a human. In one embodiment, the low dose amount refers to the use of a particular amount that is lower than typically use for immunosuppression of a human organ transplant recipient that is calculated to prevent rejection).

In certain embodiments a low dose of an immunosuppressive agent, for example Tacrolimus, is less than about ⅕, ⅙, ⅐, ⅛, ⅑, ⅒, 1/11, 1/12, 1/13, 1/14, or less then about 1/15 of a normal dose used for immunosuppression in humans. In certain embodiments, the low dose of an immunosuppressive agent, for example Tacrolimus, is about or less than about ⅒ of the amount used for immunosuppression in humans.

In other embodiments, the low dose of the immunosuppressive agent, for example Tacrolimus, is about or less than ½, ⅓, ¼, ⅕, ⅙, ⅐, ⅛, or about or less than about ⅑ of the amount used for immunosuppression in humans. In further embodiments, the low dose of the immunosuppressive agent, for example Tacrolimus, is about or less than about 0.9, 0.8, 0.7, 0.6, 0.5, 0.4, 0.3, 0.2, 0.1, 0.09, 0.08, 0.07, 0.06, 0.05, 0.04, 0.03, 0.02, 0.01, 0.009, 0.08, or 0.07 times than the typical amount used for a particular situation in humans to generate immunosuppression.

In specific embodiments, a low dose of an immunosuppressive agent (e.g., Tacrolimus) in humans is about 0.01 mg/kg to about 0.5 mg/kg, about 0.01 mg/kg to 0.5 mg/kg, about 0.01 mg/kg to about 0.45 mg/kg, about 0.01 mg/kg to about 0.4 mg/kg, about 0.01 mg/kg to about 0.35 mg/kg, about 0.06 mg/kg to about 0.45 mg/kg, about 0.07 mg/kg to about 0.4 mg/kg, about 0.08 mg/kg to about 0.35 mg/kg, about 0.09 mg/kg to about 0.3 mg/kg, about 0.1 mg/kg to about 0.25 mg/kg, and so on. In one embodiment, the low dose of Tacrolimus in humans is about 0.01 mg/kg to 0.074 mg/kg.

A normal dose of Tacrolimus for immunosuppression in humans is about 0.1 mg/kg/day-0.3 mg/kg/day (oral) and about 0.01 mg/kg/day-0.05 mg/kg/day (IV). In certain embodiments, a low dose of Tacrolimus in humans is about one tenth the normal dose; e.g., about 0.01 mg/kg/day-0.03 mg/kg/day (oral) and about 0.001 mg/kg/day-0.005 mg/kg/day (IV).

In other embodiments, a low dose of Tacrolimus in humans comprises any amount below about 0.1 mg/kg/day for oral administration. The low dose can comprise any amount below about 0.095, 0.09, 0.085, 0.08, 0.075, 0.07, 0.065, 0.06, 0.055, 0.05, 0.045, 0.04, 0.035, 0.03, 0.029, 0.028, 0.027, 0.026, 0.025, 0.024, 0.023, 0.022, 0.021, 0.020, 0.019, 0.018, 0.017, 0.016, 0.05, 0.014, 0.013, 0.012, 0.011, 0.010, 0.009, 0.008, 0.007, 0.006, 0.005, 0.004, 0.003, 0.002, or 0.001 mg/kg/day.

For intravenous administration, a low dose of Tacrolimus in humans comprises any amount below about 0.01 mg/kg/day. The low dose can comprise any amount below about 0.01, 0.009, 0.008, 0.007, 0.006, 0.005, 0.004, 0.003, 0.002, or 0.001 mg/kg/day.

In further embodiments, a low dose of Tacrolimus in humans results in a blood concentration range of about 0.01 ng/ml to about 10 ng/ml. The concentration can be less than about 10 ng/ml, 9 ng/ml, 8 ng/ml, 7 ng/ml, 6 ng/ml, 5 ng/ml, 4 ng/ml, 3 ng/ml, 2 ng/ml, 1 ng/ml, 0.9 ng/ml, 0.8 ng/ml, 0.7 ng/ml, 0.6 ng/ml, 0.5 ng/ml, 0.4 ng/ml, 0.3 ng/ml, 0.2 ng/ml, 0.1 ng/ml, 0.09 ng/ml, 0.08 ng/ml, 0.07 ng/ml, 0.06 ng/ml, 0.05 ng/ml, 0.04 ng/ml, 0.03 ng/ml, 0.02 ng/ml or 0.01 ng/ml. In another embodiment, the blood Tacrolimus concentrations after administration to humans are less than about 5 ng/ml. The concentration can range from about 0.01, 0.02, 0.03 0.04 or 0.05 ng/ml to about 1, 2, 3, 4, or 5 ng/ml, for example from about 0. 1-4 ng/ml.

In certain embodiments, the stem cell mobilizer in pharmaceutical compositions of the invention is AMD3100. In such embodiments, the pharmaceutical composition can comprise a typical human dose for AMD3100, for example about 0.12-0.24 mg/kg. In some embodiments, for a patient who has 60 kg body weight, the dosage of ADM3100 can be about 0.24 mg/kg/day by subcutaneous injection.

The pharmaceutical compositions of the invention can be formulated for substantially simultaneous administration to the subject at a single site. As used herein, “substantially simultaneous administration” means that the stem cell mobilizer and immunosuppressive agent or non-immunosuppressive FK binding ligand comprising the pharmaceutical compositions of the invention are delivered to the subject at or about the same time. For example, the stem cell mobilizer and immunosuppressive agent or non-immunosuppressive FK binding ligand can be delivered as a single formulation into a single site on the subject, or as separate formulations, for example by delivery to the same single site on a subject by successive administrations, such as successive subcutaneous or intramuscular injections, wherein the separate formulations occupy substantially the same space within the subject’s body at substantially the same time. Routes of administration where the pharmaceutical compositions of the invention are ultimately absorbed and distributed systemically, such as orally or intra-rectally, are considered as a “substantially simultaneous administration” when the stem cell mobilizer and immunosuppressive agent or non-immunosuppressive FK binding ligand are delivered in a single formulation or are delivered in separate formulations in succession.

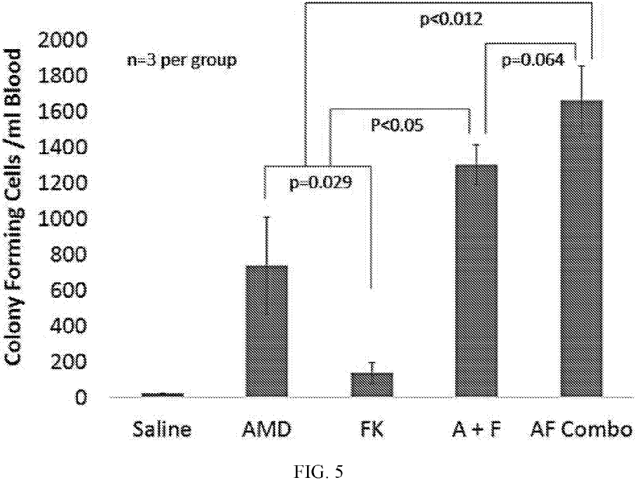

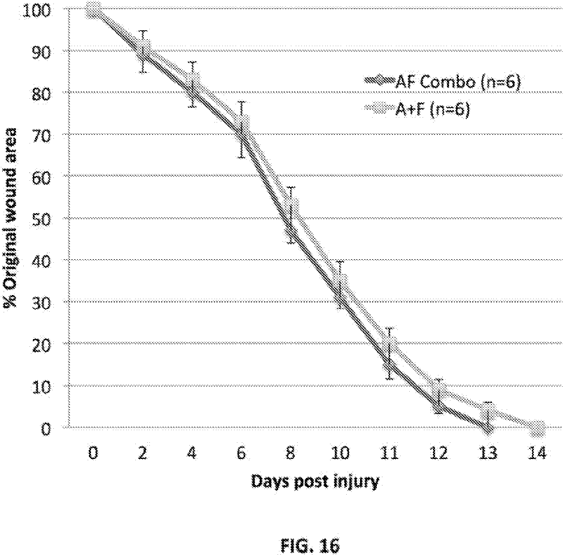

Without wishing to be bound by any specific theory, it is believed that the stem cell mobilizer and immunosuppressive agent or non-immunosuppressive FK binding ligand, when given to a subject by substantially simultaneous administration, are absorbed into the subject’s body in a way which stimulates mobilization of stem cells synergistically when compared to a stem cell mobilizer and immunosuppressive agent or non-immunosuppressive FK binding ligand administration either separately in time (whether or not at the same site on a subject) or substantially simultaneously but at different sites on a subject. This unknown and surprising synergistic effect is shown, for example, in FIG. 5 and in Example 1 below, which indicates that a single formulation of AMD3100 and Tacrolimus AF was statistically-significantly more effective in mobilizing stem cells than either AMD3100 administered alone, Tacrolimus administered alone, or AMD3100 and Tacrolimus administered separately. This synergistic effect is further shown, for example, in Example 9 below and in FIG. 16, which demonstrate that subjects treated with a pharmaceutical composition comprising a combination of AMD3100 and Tacrolimus unexpectedly had a faster wound healing time, as compared to subjects receiving AMD3100 and Tacrolimus separately (“A+F”). The synergistic combination of AMD3100 and Tacrolimus was advantageous over the A+F treatment in addition to providing faster healing time, for example in terms of administering significantly less dosages of Tacrolimus (twice as much Tacrolimus was used for the A+F treatment) which may, e.g., further avoid undesirable side effects of immunosuppression, and less overall injections were given to the subjects receiving the AMD3100 and Tacrolimus combination treatment.

Where the pharmaceutical compositions of the invention comprise the stem cell mobilizing agent AMD3100 and low dose immunosuppressive drug Tacrolimus (FK-506), in the form of combination, this combination is sometimes referred to herein as “AF” or “AF Combination.”

As described above, AMD3100 (Plerixafor or Mozobil) is a CXCR4 antagonist, which was originally developed as an anti-HIV medicine but found to potently mobilize CD34 and other stem cells from their bone marrow niche. AMD3100 was first approved by the FDA in 2008 for use in multiple myeloma cancer patients, for banking of stem cells prior to myeloablative chemotherapy. Today, AMD3100 is used, often with neupogen (G-CSF), to mobilize hematopoietic stem cells in multiple myeloma cancer patients for banking prior to myeloablative chemotherapy. These mobilized stem cells are subsequently transplanted back to the patient after cancer treatment. Thus the drug AMD3100 is well established to be safe and effective.

As described above, FK506 (Tacrolimus or Prograph) was discovered in 1987 from a type of soil bacterium, Streptomyces tsukubaensis. FK506 reduces peptidyl-prolyl isomerase activity by binding to the immunophilin FKBP12 (FK506 binding protein) creating a new complex. This FKBP12-FK506 complex interacts with and inhibits calcineurin thus inhibiting both T-lymphocyte signal transduction and IL-2 transcription. FK506 was first approved by the FDA in 1994 for use in liver transplantation, and its uses have now been extended to include kidney, heart, small bowel, pancreas, lung, trachea, skin, cornea, bone marrow and limb transplants.

The “AF” pharmaceutical compositions described herein, also called “AF Combinations,” thus provide a potent, synergistic activity of AMD3100 and low-dose Tacrolimus in mobilizing, recruiting and retaining of stem cells in the injured sites. As discussed above and as shown in FIGS. 5 and 16 and in Examples 2 and 9, the AF Combinations surprisingly show a synergistic effect in treating tissue injury as compared to the separate administration of a stem cell mobilizer (such as AMD3100) and an immunosuppressive agent (such as Tacrolimus) or FK protein binding ligand, or the simultaneous administration of a stem cell mobilizer and an immunosuppressive agent or FK protein binding ligand at different sites.

In certain embodiments, the ratio of Tacrolimus to AMD3100 is about ⅒ to 1/100 in the AF Combinations. In other embodiments, an AF Combinations comprise only two active ingredients, wherein the first active ingredient is AMD3100 and the second active ingredient is Tacrolimus, and wherein the composition comprises 10-40 mg of AMD3100 and 0.1 to 4 mg Tacrolimus. In these and other AF Combinations, the Tacrolimus enhances the potency of the AMD3100. The present pharmaceutical compositions, including AF Combinations, can thus be described in terms of a ratio of (a) an immunosuppressive drug or a FKBP ligand (including an immunosuppressive or a non-immunosuppressive FKBP ligand) to (b) a stem cell mobilizer (e.g., a CXCR antagonist). In certain embodiments, this ratio can be about 1/1, ½, ⅓, ¼, ⅕, ⅙, ⅐, ⅛, ⅑, ⅒, 1/11, 1/12, 1/13, 1/14, 1/15, 1/16, 1/17, 1/18, 1/19, 1/20, 1/21, 1/22, 1/23, 1/24, 1/25, 1/26, 1/27, 1/28, 1/29, 1/30, 1/31, 1/32, 1/33, 1/34, 1/35, 1/36, 1/37, 1/38, 1/39, 1/40, 1/41, 1/42, 1/43, 1/44, 1/45, 1/46, 1/47, 1/48, 1/49, 1/50, 1/51, 1/52, 1/53, 1/54, 1/55, 1/56, 1/57, 1/58, 1/59, 1/60, 1/61, 1/62, 1/63, 1/64, 1/65, 1/66, 1/67, 1/68, 1/69, 1/70, 1/71, 1/72, 1/73, 1/74, 1/75, 1/76, 1/77, 1/78, 1/79, 1/80, 1/81, 1/82, 1/83, 1/84, 1/85, 1/86, 1/87, 1/88, 1/89, 1/90, 1/91, 1/92, 1/93, 1/94, 1/95, 1/96, 1/97, 1/98, 1/99, 1/100, or more.

In some embodiments, the pharmaceutical compositions of the invention can comprise (a) an immunosuppressive drug or a FKBP ligand (including an immunosuppressive or a non-immunosuppressive FKBP ligand) and (b) a stem cell mobilizer in a ratio range of about ⅒-1/100, ⅒-1/99, ⅒- 1/98, ⅒-1/97, ⅒-1/96, ⅒-1/95, ⅒-1/94, ⅒-1/93, ⅒-1/92, ⅒-1/91, ⅒-1/90, ⅒-1/89, ⅒-1/88, ⅒-1/87, ⅒-1/86, ⅒-1/85, ⅒-1/84, ⅒-1/83, ⅒-1/82, ⅒- 1/81, ⅒-1/80, ⅒-1/79, ⅒-1/78, ⅒-1/77, ⅒-1/76, ⅒-1/75, ⅒-1/74, ⅒-1/73, ⅒-1/72, ⅒-1/71, ⅒-1/70, ⅒-1/69, ⅒-1/68, ⅒-1/67, ⅒-1/66, ⅒-1/65, ⅒- 1/64, ⅒-1/63, ⅒-1/62, ⅒-1/61, ⅒-1/60, ⅒-1/59, ⅒-1/58, ⅒-1/57, ⅒-1/56, ⅒-1/55, ⅒-1/54, ⅒-1/53, ⅒-1/52, ⅒-1/51, ⅒-1/50, ⅒-1/49, ⅒-1/48, ⅒- 1/47, ⅒-1/46, ⅒-1/45, ⅒-1/44, ⅒-1/43, ⅒-1/42, ⅒-1/41, ⅒-1/40, ⅒-1/39, ⅒-1/38, ⅒-1/37, ⅒-1/36, ⅒-1/35, ⅒-1/34, ⅒-1/33, ⅒-1/32, ⅒-1/31, ⅒- 1/30, ⅒-1/29, ⅒-1/28, ⅒-1/27, ⅒-1/26, ⅒-1/25, ⅒-1/24, ⅒-1/23, ⅒-1/22, ⅒-1/21, ⅒-1/20, ⅒-1/19, ⅒-1/18, ⅒-1/17, ⅒-1/16, ⅒-1/15, ⅒-1/14, ⅒- 1/13, ⅒-1/12, or ⅒-1/11.

In other embodiments, the pharmaceutical compositions can comprise (a) an immunosuppressive drug or a FKBP ligand (including an immunosuppressive or a non-immunosuppressive FKBP ligand) and (b) a stem cell mobilizer in a ratio range of about 1/15-1/100, 1/15-1/99, 1/15-1/98, 1/15-1/97, 1/15-1/96, 1/15-1/95, 1/15-1/94, 1/15-1/93, 1/15-1/92, 1/15-1/91, 1/15-1/90, 1/15-1/89, 1/15-1/88, 1/15-1/87, 1/15-1/86, 1/15-1/85, 1/15- 1/84, 1/15-1/83, 1/15-1/82, 1/15-1/81, 1/15-1/80, 1/15-1/79, 1/15-1/78, 1/15-1/77, 1/15-1/76, 1/15-1/75, 1/15-1/74, 1/15-1/73, 1/15-1/72, 1/15-1/71, 1/15-1/70, 1/15-1/69, 1/15-1/68, 1/15- 1/67, 1/15-1/66, 1/15-1/65, 1/15-1/64, 1/15-1/63, 1/15-1/62, 1/15-1/61, 1/15-1/60, 1/15-1/59, 1/15-1/58, 1/15-1/57, 1/15-1/56, 1/15-1/55, 1/15-1/54, 1/15-1/53, 1/15-1/52, 1/15-1/51, 1/15- 1/50, 1/15-1/49, 1/15-1/48, 1/15-1/47, 1/15-1/46, 1/15-1/45, 1/15-1/44, 1/15-1/43, 1/15-1/42, 1/15-1/41, 1/15-1/40, 1/15-1/39, 1/15-1/38, 1/15-1/37, 1/15-1/36, 1/15-1/35, 1/15-1/34, 1/15- 1/33, 1/15-1/32, 1/15-1/31, 1/15-1/30, 1/15-1/29, 1/15-1/28, 1/15-1/27, 1/15-1/26, 1/15-1/25, 1/15-1/24, 1/15-1/23, 1/15-1/22, 1/15-1/21, 1/15-1/20, 1/15-1/19, 1/15-1/18, 1/15-1/17, or 1/15-1/16.

In some embodiments, the ratio range of (a) an immunosuppressive drug or a FKBP ligand (including an immunosuppressive or a non-immunosuppressive FKBP ligand) to (b) a stem cell mobilizer within a pharmaceutical composition of the invention can comprise about 1/20-1/100, 1/20- 1/99, 1/20-1/98, 1/20-1/97, 1/20-1/96, 1/20-1/95, 1/20-1/94, 1/20-1/93, 1/20-1/92, 1/20-1/91, 1/20-1/90, 1/20-1/89, 1/20-1/88, 1/20-1/87, 1/20-1/86, 1/20-1/85, 1/20-1/84, 1/20-1/83, 1/20- 1/82, 1/20-1/81, 1/20-1/80, 1/20-1/79, 1/20-1/78, 1/20-1/77, 1/20-1/76, 1/20-1/75, 1/20-1/74, 1/20-1/73, 1/20-1/72, 1/20-1/71, 1/20-1/70, 1/20-1/69, 1/20-1/68, 1/20-1/67, 1/20-1/66, 1/20- 1/65, 1/20-1/64, 1/20-1/63, 1/20-1/62, 1/20-1/61, 1/20-1/60, 1/20-1/59, 1/20-1/58, 1/20-1/57, 1/20-1/56, 1/20-1/55, 1/20-1/54, 1/20-1/53, 1/20-1/52, 1/20-1/51, 1/20-1/50, 1/20-1/49, 1/20- 1/48, 1/20-1/47, 1/20-1/46, 1/20-1/45, 1/20-1/44, 1/20-1/43, 1/20-1/42, 1/20-1/41, 1/20-1/40, 1/20-1/39, 1/20-1/38, 1/20-1/37, 1/20-1/36, 1/20-1/35, 1/20-1/34, 1/20-1/33, 1/20-1/32, 1/20- 1/31, 1/20-1/30, 1/20-1/29, 1/20-1/28, 1/20-1/27, 1/20-1/26, 1/20-1/25, 1/20-1/24, 1/20-1/23, 1/20-1/22, or 1/20-1/21.

In other embodiments, the ratio range of (a) an immunosuppressive drug or a FKBP ligand (including an immunosuppressive or a non-immunosuppressive FKBP ligand) to (b) a stem cell mobilizer within a pharmaceutical composition of the invention can comprise about 1/30- 1/100, 1/30-1/99, 1/30-1/98, 1/30-1/97, 1/30-1/96, 1/30-1/95, 1/30-1/94, 1/30-1/93, 1/30-1/92, 1/30-1/91, 1/30-1/90, 1/30-1/89, 1/30-1/88, 1/30-1/87, 1/30-1/86, 1/30-1/85, 1/30-1/84, 1/30-1/83, 1/30-1/82, 1/30-1/81, 1/30-1/80, 1/30-1/79, 1/30-1/78, 1/30-1/77, 1/30-1/76, 1/30-1/75, 1/30-1/74, 1/30-1/73, 1/30-1/72, 1/30-1/71, 1/30-1/70, 1/30-1/69, 1/30-1/68, 1/30-1/67, 1/30-1/66, 1/30-1/65, 1/30-1/64, 1/30-1/63, 1/30-1/62, 1/30-1/61, 1/30-1/60, 1/30-1/59, 1/30-1/58, 1/30-1/57, 1/30-1/56, 1/30-1/55, 1/30-1/54, 1/30-1/53, 1/30-1/52, 1/30-1/51, 1/30-1/50, 1/30-1/49, 1/30-1/48, 1/30-1/47, 1/30-1/46, 1/30-1/45, 1/30-1/44, 1/30-1/43, 1/30-1/42, 1/30-1/41, 1/30-1/40, 1/30-1/39, 1/30-1/38, 1/30-1/37, 1/30-1/36, 1/30-1/35, 1/30-1/34, 1/30-1/33, 1/30-1/32, or 1/30-1/31.

In further embodiments, the pharmaceutical compositions of the invention can comprise (a) an immunosuppressive drug or a FKBP ligand (including an immunosuppressive or a non-immunosuppressive FKBP ligand) and (b) a stem cell mobilizer in a ratio range of about 1/40-1/100, 1/40-1/99, 1/40-1/98, 1/40-1/97, 1/40-1/96, 1/40-1/95, 1/40-1/94, 1/40-1/93, 1/40-1/92, 1/40-1/91, 1/40-1/90, 1/40-1/89, 1/40-1/88, 1/40-1/87, 1/40-1/86, 1/40-1/85, 1/40-1/84, 1/40-1/83, 1/40-1/82, 1/40-1/81, 1/40-1/80, 1/40-1/79, 1/40-1/78, 1/40-1/77, 1/40-1/76, 1/40-1/75, 1/40-1/74, 1/40-1/73, 1/40-1/72, 1/40-1/71, 1/40-1/70, 1/40-1/69, 1/40-1/68, 1/40-1/67, 1/40-1/66, 1/40-1/65, 1/40-1/64, 1/40-1/63, 1/40-1/62, 1/40-1/61, 1/40-1/60, 1/40-1/59, 1/40-1/58, 1/40-1/57, 1/40-1/56, 1/40-1/55, 1/40-1/54, 1/40-1/53, 1/40-1/52, 1/40-1/51, 1/40-1/50, 1/40-1/49, 1/40-1/48, 1/40-1/47, 1/40-1/46, 1/40-1/45, 1/40-1/44, 1/40-1/43, 1/40-1/42, or 1/40-1/41.

In other embodiments, the pharmaceutical compositions of the invention can comprise (a) an immunosuppressive drug or a FKBP ligand (including an immunosuppressive or a non-immunosuppressive FKBP ligand) and a stem cell mobilizer in a ratio range of about 1/50-1/100, 1/50-1/99, 1/50-1/98, 1/50-1/97, 1/50-1/96, 1/50-1/95, 1/50-1/94, 1/50-1/93, 1/50- 1/92, 1/50-1/91, 1/50-1/90, 1/50-1/89, 1/50-1/88, 1/50-1/87, 1/50-1/86, 1/50-1/85, 1/50-1/84, 1/50-1/83, 1/50-1/82, 1/50-1/81, 1/50-1/80, 1/50-1/79, 1/50-1/78, 1/50-1/77, 1/50-1/76, 1/50-1/75, 1/50-1/74, 1/50-1/73, 1/50-1/72, 1/50-1/71, 1/50-1/70, 1/50-1/69, 1/50-1/68, 1/50-1/67, 1/50-1/66, 1/50-1/65, 1/50-1/64, 1/50-1/63, 1/50-1/62, 1/50-1/61, 1/50-1/60, 1/50-1/59, 1/50- 1/58, 1/50-1/57, 1/50-1/56, 1/50-1/55, 1/50-1/54, 1/50-1/53, 1/50-1/52, 1/50-1/51, 1/60- 1/100, 1/60-1/99, 1/60-1/98, 1/60-1/97, 1/60-1/96, 1/60-1/95, 1/60-1/94, 1/60-1/93, 1/60- 1/92, 1/60-1/91, 1/60-1/90, 1/60-1/89, 1/60-1/88, 1/60-1/87, 1/60-1/86, 1/60-1/85, 1/60-1/84, 1/60-1/83, 1/60-1/82, 1/60-1/81, 1/60-1/80, 1/60-1/79, 1/60-1/78, 1/60-1/77, 1/60-1/76, 1/60- 1/75, 1/60-1/74, 1/60-1/73, 1/60-1/72, 1/60-1/71, 1/60-1/70, 1/60-1/69, 1/60-1/68, 1/60-1/67, 1/60-1/66, 1/60-1/65, 1/60-1/64, 1/60-1/63, 1/60-1/62, 1/60-1/61,

In other embodiments, the ratio range of (a) an immunosuppressive drug or a FKBP ligand (including an immunosuppressive or a non-immunosuppressive FKBP ligand) to (b) a stem cell mobilizer within a pharmaceutical composition of the invention can comprise about 1/70- 1/100, 1/70-1/99, 1/70-1/98, 1/70-1/97, 1/70-1/96, 1/70-1/95, 1/70-1/94, 1/70-1/93, 1/70-1/92, 1/70-1/91, 1/70-1/90, 1/70-1/89, 1/70-1/88, 1/70-1/87, 1/70-1/86, 1/70-1/85, 1/70-1/84, 1/70-1/83, 1/70-1/82, 1/70-1/81, 1/70-1/80, 1/70-1/79, 1/70-1/78, 1/70-1/77, 1/70-1/76, 1/70-1/75, 1/70-1/74, 1/70-1/73, 1/70-1/72, 1/70-1/71, 1/80-1/100, 1/80-1/99, 1/80-1/98, 1/80-1/97, 1/80-1/96, 1/80-1/95, 1/80-1/94, 1/80-1/93, 1/80-1/92, 1/80-1/91, 1/80-1/90, 1/80-1/89, 1/80-1/88, 1/80-1/87, 1/80-1/86, 1/80-1/85, 1/80-1/84, 1/80-1/83, 1/80-1/82, 1/80-1/81, 1/90-1/100, 1/90-1/99, 1/90-1/98, 1/90-1/97, 1/90-1/96, 1/90-1/95, 1/90-1/94, 1/90-1/93, 1/90- 1/92, or 1/90-1/91.

In certain embodiments, the pharmaceutical compositions of the invention comprise (a) a non-immunosuppressive FKBP ligand and (b) a stem cell mobilizer (e.g., a CXCR antagonist). In such embodiments, the ratio of non-immunosuppressive FKBP ligand to stem cell mobilizer can be between about ⅒ to 1/100. In further embodiments, the ratio can be greater than about ⅒ including about 1/1, ½, ⅓, ¼, ⅕, ⅙, ⅐, ⅛ or ⅑. In other embodiments, the ratio can be less than about 1/100 including, but not limited to, about 1/150, 1/200, 1/250, 1/300, 1/350, 1/400, 1/450, and 1/500 or more (including ranges of the foregoing).

In certain embodiments, the pharmaceutical compositions of the invention, including AF Combinations, may be administered at least once a week over the course of several weeks. In one embodiment, the pharmaceutical compositions are administered at least once a week over several weeks to several months. In another embodiment, the pharmaceutical compositions are administered once a week over four to eight weeks. In yet another embodiment, the pharmaceutical compositions are administered once a week over four weeks.

In other embodiments, the present pharmaceutical compositions, including AF Combinations, can be administered at least once a day for about 2 days, at least once a day for about 3 days, at least once a day for about 4 days, at least once a day for about 5 days, at least once a day for about 6 days, at least once a day for about 7 days, at least once a day for about 8 days, at least once a day for about 9 days, at least once a day for about 10 days, at least once a day for about 11 days, at least once a day for about 12 days, at least once a day for about 13 days, at least once a day for about 14 days, at least once a day for about 15 days, at least once a day for about 16 days, at least once a day for about 17 days, at least once a day for about 18 days, at least once a day for about 19 days, at least once a day for about 20 days, at least once a day for about 21 days, at least once a day for about 22 days, at least once a day for about 23 days, at least once a day for about 24 days, at least once a day for about 25 days, at least once a day for about 26 days, at least once a day for about 27 days, at least once a day for about 28 days, at least once a day for about 29 days, at least once a day for about 30 days, or at least once a day for about 31 days.

In other embodiments, the pharmaceutical compositions of the invention, including AF Combinations, can be administered every other day for about 2 days, every other day for about 3 days, every other day for about 4 days, every other day for about 5 days, every other day for about 6 days, every other day for about 7 days, every other day for about 8 days, every other day for about 9 days, every other day for about 10 days, every other day for about 11 days, every other day for about 12 days, every other day for about 13 days, every other day for about 14 days, every other day for about 15 days, every other day for about 16 days, every other day for about 17 days, every other day for about 18 days, every other day for about 19 days, every other day for about 20 days, every other day for about 21 days, every other day for about 22 days, every other day for about 23 days, every other day for about 24 days, every other day for about 25 days, every other day for about 26 days, every other day for about 27 days, every other day for about 28 days, every other day for about 29 days, every other day for about 30 days, or every other day for about 31 days or more.

In other embodiments, the pharmaceutical compositions of the invention, including AF Combinations, can be administered about once every day, about once every 2 days (also sometimes stated herein as once every other day), about once every 3 days, about once every 4 days, about once every 5 days, about once every 6 days, about once every 7 days, about once every 8 days, about once every 9 days, about once every 10 days, about once every 11 days, about once every 12 days, about once every 13 days, about once every 14 days, about once every 15 days, about once every 16 days, about once every 17 days, about once every 18 days, about once every 19 days, about once every 20 days, about once every 21 days, about once every 22 days, about once every 23 days, about once every 24 days, about once every 25 days, about once every 26 days, about once every 27 days, about once every 28 days, about once every 29 days, about once every 30 days, or about once every 31 days. In certain embodiments, the present pharmaceutical compositions can be administered every other day.

In other embodiments, the pharmaceutical compositions of the invention, including AF Combinations, can be administered about once every week, about once every 2 weeks, about once every 3 weeks, about once every 4 weeks, about once every 5 weeks, about once every 6 weeks, about once every 7 weeks, about once every 8 weeks, about once every 9 weeks, about once every 10 weeks, about once every 11 weeks, about once every 12 weeks, about once every 13 weeks, about once every 14 weeks, about once every 15 weeks, about once every 16 weeks, about once every 17 weeks, about once every 18 weeks, about once every 19 weeks, or about once every 20 weeks.

In other embodiments, the pharmaceutical compositions of the invention, including AF Combinations, can be administered about once every month, about once every 2 months, about once every 3 months, about once every 4 months, about once every 5 months, about once every 6 months, about once every 7 months, about once every 8 months, about once every 9 months, about once every 10 months, about once every 11 months, or about once every 12 months.

In other embodiments, the pharmaceutical compositions of the invention, including AF Combinations, can be administered at least once a week for about 2 weeks, at least once a week for about 3 weeks, at least once a week for about 4 weeks, at least once a week for about 5 weeks, at least once a week for about 6 weeks, at least once a week for about 7 weeks, at least once a week for about 8 weeks, at least once a week for about 9 weeks, at least once a week for about 10 weeks, at least once a week for about 11 weeks, at least once a week for about 12 weeks, at least once a week for about 13 weeks, at least once a week for about 14 weeks, at least once a week for about 15 weeks, at least once a week for about 16 weeks, at least once a week for about 17 weeks, at least once a week for about 18 weeks, at least once a week for about 19 weeks, or at least once a week for about 20 weeks.

In other embodiments, the pharmaceutical compositions of the invention, including AF Combinations, can be administered at least once a week for about 1 month, at least once a week for about 2 months, at least once a week for about 3 months, at least once a week for about 4 months, at least once a week for about 5 months, at least once a week for about 6 months, at least once a week for about 7 months, at least once a week for about 8 months, at least once a week for about 9 months, at least once a week for about 10 months, at least once a week for about 11 months, or at least once a week for about 12 months.

In certain embodiments, the pharmaceutical compositions of the invention, including AF Combinations, can be administered in a dosing regimen or treatment method comprising administering the pharmaceutical composition to a subject who is suffering from a tissue injury in one or more doses at the time a tissue injury is incurred or observed, and at about one month, about two months and about three months after a tissue injury is incurred or observed, for a total of four administrations. Any suitable administration route can be used. In some embodiments of the dosing regimen or treatment method, the administration route used is subcutaneous or intramuscular. In other embodiments of the dosing regimen or treatment method, the administration route is subcutaneous. In certain embodiments, the one or more doses can be administered about when the tissue injury is incurred or observed and at about one month, about two months and about three months after a tissue injury is incurred or observed can comprise one dose, two doses, three doses, four doses, five doses, six doses, seven doses, eight doses, nine doses or ten doses. The one or more doses can be administered over the course of one or more days from when the tissue injury is incurred or observed and at about one month, about two months and about three months after a tissue injury, for example one dose every day or every other day. The one or more doses can be administered in evenly- or unevenly-spaced intervals from when the tissue injury is incurred or observed and at about one month, about two months and about three months after a tissue injury. In one embodiment, the one or more doses can be administered on about the day the tissue injury was incurred or observed (day zero), and again at about days 2, 4, 6, and 8 after the day the tissue injury was incurred or observed; about on the one-month anniversary of the day the tissue injury was incurred or observed and on about days 2, 4, 6 and 8 after the one-month anniversary of the day the tissue injury was incurred or observed; again on about the two-month anniversary of the day the tissue injury was incurred or observed and on about days 2, 4, 6 and 8 after the two-month anniversary of the day the tissue injury was incurred or observed; and again on about the three-month anniversary of the day the tissue injury was incurred or observed and on about days 2, 4, 6 and 8 after the three-month anniversary of the day the tissue injury was incurred or observed.

In other embodiments, the pharmaceutical compositions of the invention, including AF Combinations, can be administered in a dosing regimen or treatment method comprising administering the pharmaceutical composition every other day to a subject who is suffering from a tissue injury. Any suitable administration route can be used, for example subcutaneous or intramuscular administration. In certain embodiments, the administration route is subcutaneous. Dosing of the present pharmaceutical compositions every other day can be continued until the tissue injury has been treated, and/or can be continued for a predetermined period of time, for example about one week, 8 days, 9 days, 10 days, 11 days, 12 days, 13 days, 2 weeks, 3 weeks, 4 weeks, 29 days, 30 days, 5 weeks or six weeks.

In treatment methods of the invention, the tissue injury which triggers administration of the first dose of the present pharmaceutical compositions, including the first does of AF Combinations, can be any tissue injury that signals or indicates that a particular treatment is necessary. For example, the tissue injury can be an organ transplant (including liver, heart, lung, kidney or corneal transplant or a skin graft), the occurrence of a burn, wound, nerve injury and/or degeneration (including spinal cord injury), or the diagnosis of IBD or other autoimmune or inflammatory disease or the occurrence of an episode of IBD or other autoimmune or inflammatory disease. For example, administration of the first dose can be immediately upon occurrence of the tissue injury, or as soon thereafter as is practical or medically feasible, for example on the same day that the tissue injury occurred or was observed, such as within about one minute, five minutes, thirty minutes, sixty minutes, ninety minutes, 2 hours, 3 hours, 4 hours, 5 hours, 10 hours, 12 hours or 18 hours after occurrence of the tissue injury. In some embodiments, administration of the first dose can be delayed from the occurrence of the tissue injury, for example by about one day, 2 days, 3 days, 4 days, 5 days, 6 days or 7 days.

Any type of burn or wound can be treated with the pharmaceutical compositions and methods of the invention (including AF Combinations), including lacerations, tears, abrasions, punctures or combinations of these. Wounds that can be treated by the pharmaceutical compositions and methods of the invention can be generated by any source, such as by physical means (e.g., accident, inflicted by self or others, surgical intervention, etc.) or can be generated as sequelae to a disease, disorder or condition such as diabetes or immobility. Burns that can be treated by the pharmaceutical compositions and methods of the invention can be generated by any source, for example by exposure of skin or other tissue to extreme heat or cold.

Large full-thickness burns and soft tissue injuries continue to pose significant surgical and medical challenges in both military and civilian injuries, due to limitations of autogenous skin, wound infection, severe metabolic stress and other associated injuries. Human deceased donor skin allografts represent a suitable and much used temporizing option for skin cover following severe bum injury. However, graft rejection is common once the immune suppressive effect of the bum has subsided, and post-burn scars generally occur. Hypertrophic scarring is also extremely common, and is the source of most morbidity related to burns. The inventor has now discovered that the present pharmaceutical compositions, including AF Combinations, and methods can mobilize populations of autochthonous stem cells and induce host repopulation of skin allografts. This conversion to chimeric skin permits extended graft acceptance, or “take,” without the need for immunosuppression (see, e.g., Example 2 below).

In one embodiment, the invention provides a method of treating full-thickness burns or soft tissue injuries in a subject, comprising administering a pharmaceutical composition of the invention, such as AF Combinations, every other day, for example beginning on the day the bum or wound is incurred for a predetermined period of time or until the burn or wound is substantially healed.

Diabetes affects nearly two hundred million people worldwide, and a great number of diabetics may have decrease wound-healing ability. Among patients with diabetes, 15% may also develop wounds such as a foot ulcer, and 12-24% of individuals with such foot ulcers may require amputation. Healing a diabetic foot ulcer or other wound more quickly can limit the complications that may lead to lower extremity amputation, morbidity and mortality in the diabetic subject. However, diabetic foot ulcers and other wounds suffered by diabetics are generally hard to heal. The inventor has now discovered that the present pharmaceutical compositions, including AF Combinations, and methods can enhance the healing of diabetic wounds, such as foot ulcers (see, e.g., Example 3 below).

In one embodiment, the invention provides a method of treating wounds on a diabetic subject, for example diabetic ulcers (including diabetic foot ulcers) comprising administering a pharmaceutical composition of the invention, such as AF Combinations, every other day, for example beginning on the day the wound is incurred or the ulcer is observed for a predetermined amount of time or until the wound is substantially healed.

Inflammatory bowel disease (IBD), including ulcerative colitis and Crohn’s disease, is a chronic relapsing disease that leads to structural damage with destruction of the bowel wall. These conditions are characterized not only by the sub-mucosal accumulation of inflammatory cells, but also by the severe damage to the epithelial layer. Based on the idea that IBD is initiated and maintained by spontaneous development of mucosal inflammation, current treatment approaches are predominately aimed at suppressing overt inflammation and include the use of pharmacological agents (corticosteroids and immune-modulators), biologics (anti-TNF-alpha), and surgery to remove sections of inflamed bowel. However, these treatment modalities have their limitations, in part due to patient non-adherence and relapse. Moreover, approximately ⅓ of IBD patients do not respond to any given therapy, and there is no cure for IBD. Currently, most IBD therapies in development are antibody-based biologics, which all have the potential for loss of therapeutic response due to the generation of antibodies to the biologics, sometimes called anti-drug antibodies or ADA.

Recent clinical studies have featured “mucosal healing” as the most significant prognostic factor for long-term remission in IBD patients, suggesting that accomplishment of epithelial regeneration is critically required to improve the treatment for IBD. A regenerative medicine approach using cell-based therapies is currently viewed as one of the most promising options for the curative treatment of IBD. Stem cells are the focus of many applications in regenerative medicine because of their extensive ability to self-renew and to generate differentiated progeny cells. Mesenchymal stromal cells (MSCs) are attractive for cell therapy due to their immunomodulatory and regenerative properties and robust in vitro proliferative capacity. Autologous and allogeneic adipose- or bone marrow-derived sources of MSCs have therefore been utilized in the early phase clinical trials for the treatment of IBD. Despite the encouraging results of recent clinical trials employing stem cell-based therapies as treatment for IBD, the complex, time consuming and expensive process needed to harvest, expand and transplant the cells makes it difficult to treat large numbers of patients. The inventor has now discovered that the present pharmaceutical compositions, including AF Combinations, and methods can treat autoimmune or inflammatory diseases such as IBD (including colitis and Crohn’s disease) by mobilizing autochthonous stem cells to the site of inflammation and/or damage in the gut (see, e.g., Example 5 below).

In one embodiment, the invention provides a method of treating an autoimmune disease or disorder in a subject, for example IBD (including colitis or Crohn’s disease), comprising administering a pharmaceutical composition of the invention, such as AF Combinations, every other day, for example beginning on the day the autoimmune disease or disorder is diagnosed or symptoms or an episode related to the autoimmune disease or disorder is observed, until the autoimmune disease or disorder is treated, or the symptoms or episode related to the autoimmune disease or disorder, is treated or for a predetermined time, such as three weeks.

The incidence of spinal cord injury (SCI) in the United States is more than 10,000 per year, resulting in 720 per million persons and enduring permanent disability each year. SCI involves impairment in motor and/or sensory function, which is characterized by the rapid development of a necrotic core of damaged tissue at the site of injury, followed by the long delayed secondary degeneration. This secondary degeneration lasts over weeks or months and is accompanied by chronic progressive destruction of resident cells, including oligodendrocytes, and further demyelination of neuropathways. To date, no proven therapeutic modality exists that has demonstrated a positive effect in neurologic outcome for SCI.

Advances in stem cell biology in the last decade have shown that stem cells might provide a good source of neurons and glia, as well as exerting a neuroprotective effect on the host tissue, thus opening new horizons for tissue engineering and regenerative medicine. Despite the encouraging results of recent animal studies and clinical trials employing stem cell-based therapies as treatment for SCI, the recovery is not complete and the facilitated repair of the spinal cord still remains insufficient. Moreover, the complex, time consuming and expensive process needed to harvest, expand and transplant endogenous cells makes it difficult to effectively treat large numbers of patients. The AF combination is attractive for therapy of SCI due to its anti-inflammatory and regenerative properties. The inventor has now discovered that the present pharmaceutical compositions, including AF Combinations, and methods can treat SCI by mobilizing autochthonous stem cells to the site of damage (see, e.g., Example 6 below).

In one embodiment, the invention provides a method of treating SCI, including acute injury and secondary degradation of spinal nerves, in a subject, comprising administering a pharmaceutical composition of the invention, such as AF Combinations, every other day, for example beginning on the day the SCI is incurred, until the SCI, or the symptoms related to the SCI, is treated, or for a predetermined time, such as 29 or 30 days after occurrence of the SCI. In some embodiments, administration of the a pharmaceutical composition of the invention, such as AF Combinations, can be delayed for a period of time after occurrence of the SCI, for example by one day or five days after occurrence of the SCI.

Anyone practicing the treatment methods of the invention to treat a tissue injury can readily determine whether the administration of the present pharmaceutical compositions is treating the tissue injury using well-known techniques or knowledge. For example, the healing of wounds or burns can be visually observed by periodic monitoring. Treatment of autoimmune diseases or disorders, such as IBD, can be determined by monitoring the subject for changes in the degree of inflammation in the affected regions, the lessening of related symptoms, and/or changes in the levels of biomarkers (such as inflammatory cytokines or autoantibodies) in the blood or in tissues. Treatment of SCI can be determined by monitoring the subject over time for the return of sensation to the affected parts of the body or by observing changes in motor function.

Without further elaboration, it is believed that one skilled in the art, using the preceding description, can utilize the present invention to the fullest extent. The following examples are illustrative only, and not limiting of the remainder of the disclosure in any way whatsoever.

### EXAMPLES

The following examples are put forth so as to provide those of ordinary skill in the art with a complete disclosure and description of how the compounds, compositions, articles, devices, and/or methods described and claimed herein are made and evaluated, and are intended to be purely illustrative and are not intended to limit the scope of what the inventors regard as their invention. Efforts have been made to ensure accuracy with respect to numbers (e.g., amounts, temperature, etc.) but some errors and deviations should be accounted for herein. Unless indicated otherwise, parts are parts by weight, temperature is in degrees Celsius or is at ambient temperature, and pressure is at or near atmospheric. There are numerous variations and combinations of reaction conditions, e.g., component concentrations, desired solvents, solvent mixtures, temperatures, pressures and other reaction ranges and conditions that can be used to optimize the product purity and yield obtained from the described process.

### Materials and Methods for Examples 1 and 2

### Injectable AF Combination

Reagents: AMD3100 and Tacrolimus (powder) were obtained from Sigma-Aldrich (St. Louis, MO).

Tacrolimus formulation: Because the hydrophobic nature of Tacrolimus powder and its poor solubility in water solutions (e.g., saline), Tacrolimus powder was dissolved in a mixture of 100% ethanol (8% of total volume), castor oil (2% of total volume) and sterile saline for injections (90% of total volume. Tacrolimus has an empirical formula of C₄₄H₆₉NO₁₂•H₂O and a formula weight of 822.03.

AMD3100 formulation: 24 mg AMD3100 was dissolved in sterilized water containing 5.9 mg of sodium chloride, and adjust to a pH of 6.0 to 7.5 with hydrochloric acid and with sodium hydroxide, if required. The molecular weight of AMD3100 is 502.79 g/mol.

AF Combination: solubilized Tacrolimus was added into AMD3100 solution according to the weight ratio of Tacrolimus to AMD3100 at about 1:10 to 1:100. For example, 0.2 mg/0.2 ml FK506 was added into 20 mg/0.8 ml AMD3100 to make 1ml composition containing 20 mg AMD3100 and 0.2 mg FK506. In a patient who has 60 kg body weight, the dosage of AMD3100 is about 0.24 mg/kg/day; therefore, 0.72ml composition is administered to the patient which includes 14.4 mg AMD3100 and 0.144 mg Tacrolimus (0.0024 mg/kg).

### Example 1 - Assay for Stem Cell Mobilization Activity by the AF Combination

Hematopoietic colony-forming cell (CFC) assays have been used extensively for research and clinical applications in humans and animal models as a way of quantifying and assessing the hematopoietic progenitor content of a cell sample. The stem cell mobilization activity can be measured by using CFC assays in mobilized peripheral blood samples. CFCs are able to divide and differentiate into a colony of more mature cells that can be detected by light microscopy. This allows for the quantification of multipotent stem cell lineages mobilized by pharmaceutical agents.

C57/B6 mice were divided into four treatment groups: 1) control group treated with saline; 2) AMD3100 group (1.0 mg/kg); (3) Tacrolimus group treated with

low-dose (0.1 mg/kg); and (4) AF Combination (containing AMD3100 and Tacrolimus). Animals were sacrificed at 3 hours after drug treatment, peripheral blood was collected and peripheral blood mononuclear cells (PBMC) were isolated. Viable PBMCs were counted and mixed with Methocult GF methylcellulose medium (StemCell Technologies, Vancouver, BC, Canada; Cat.: 03444) containing 100 Uml penicillin and 100 µg/ml streptomycin to give a final density of 1×105 viable PBMCs/1.5 ml of medium. The Methocult-cell mixture was dispensed into Ultra Low Cell Adherence Surface six-well plates (Coming, Lowell, MA), and cultures were incubated at 37° C., 5% CO2, and 95% humidity for 14 days. Colony-forming units (CFUs) were counted using an inverted microscope (Carl Zeiss Microscopy, Thornwood, NY) at X 4. The number of CFUs was determined by dividing the number of viable cells per milliliter by the number of plated cells per well and multiplying by the number of counted CFUs/well.

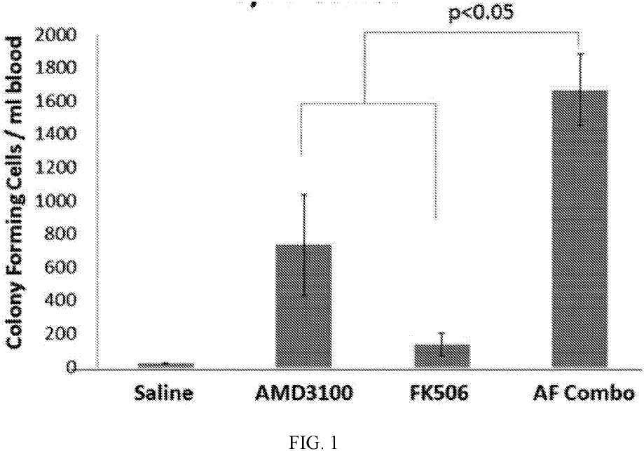

FIG. 1 shows that the number of colony forming cells (CFCs) was significantly increased in mice treated with AMD3100 or Tacrolimus alone. Surprisingly, the number of CFCs was even higher when mice were treated with AF Combination. These results indicate a potent synergistic activity of the components in the AF Combination in mobilization of endogenous stem cells. See also FIG. 5, which shows that the AF Combination was more effective in mobilizing stem cells than either AMD3100 administered alone, Tacrolimus administered alone, or the A+F dual drug treatment (that is, stem cell mobilizer AMD3100 and the immunosuppressive agent Tacrolimus administered separately).

### Example 2 - Improved Burn Wound Healing by AF Combination in Mice

Stem cell therapy can improve the quality of burn wound healing, reduce the formation of scars and re-establish the skin. To avoid the need for a preparation of endogenous stem cells to treat burns, which is expensive and time-consuming, this study shows that endogenous bone marrow stem cells can be mobilized pharmacologically with AF Combination to treat burns.

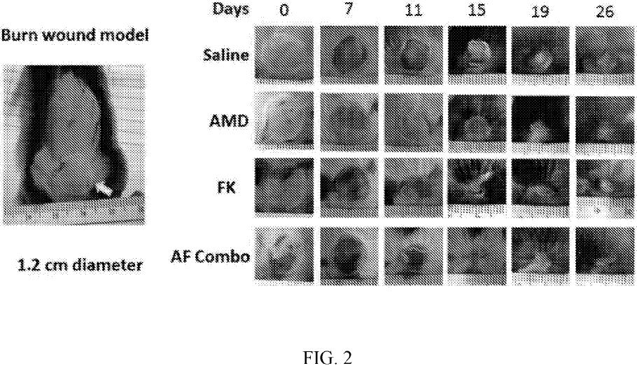

Full-thickness burns (12 mm in diameter) were created in the dorsal skin of C57/B6 mice (FIG. 2). Burned mice were divided randomly into four experimental groups as follows and received subcutaneous injections of saline or drugs immediately after wounding until complete healing: (1) control group treated with saline; (2) AMD3100 group treated every other day (1.0 mg/kg); (3) FK506 group treated daily with low-dose (0.1 mg/kg); and (4) AF Combination group treated every other day. All wound evaluations were double blinded.

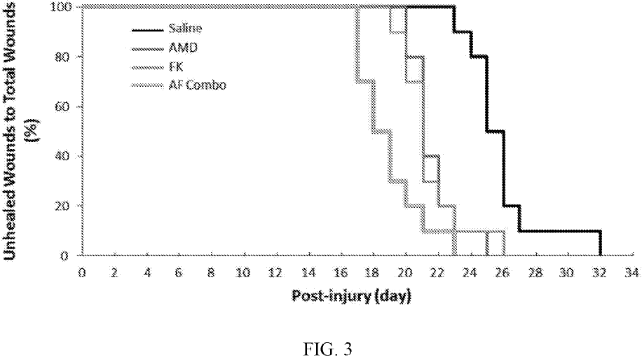

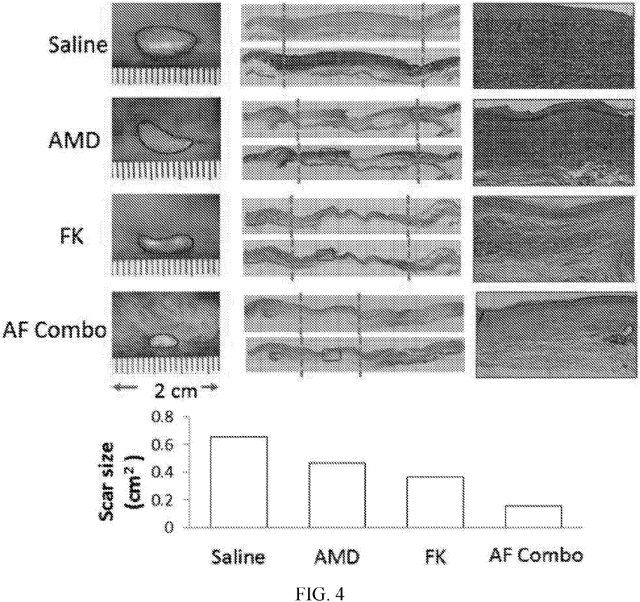

Endogenous stem cell mobilization produced by AF Combination was able to reduce by 25% the time of complete healing of full-thickness wounds (19±2 days versus 26±3 days, n=10/group, p<0.001) (FIG. 3, which was accompanied by reduced scar formation assessed both macroscopically and histologically (FIG. 4). These results indicate that AF Combination mobilized an increased number of lineage-negative CD133+, c-Kit+, CXCR4+ stem cells and M2 macrophages in the burn sites at 7 days after wounding. AF Combination treatment also increased the expression of stromal cell-derived factor (SDF)-1 and angiogenic cytokines (VEGF, b-FGF, HGF) in the granulation tissues. Lineage-tracing studies with CD133+/C-L mice containing Rosa26GFP reporter allele further demonstrated the contributions of CD133+ cells to the improved repair process in dual-treated bum wounds.

In conclusion, mobilization, recruitment, and retention of endogenous stem cells with AF Combination results in better and faster healing of burn wounds. These findings offer a significantly advantageous therapeutic approach to bum wound healing.

### Materials and Methods for Examples 3 Through 7

The AF Combination drug composition (AMD3100 + FK506) was prepared from FK506 powder (Sigma) and AMD3100 powder (Sigma), both of which were stored at -20⁰C prior to use in preparing the solutions. The final prepared solutions (Composition I and Composition II; see below) were stored at 4 ⁰C until used.

### Composition I (Used for Rodents Such as Rats and Mice)

FK506

$$\begin{matrix}\text{Stock solution} \left(12 \text{mg} / \text{ml}\right) = \\ 120 \text{mg FK powder + 5ml 95\% EtOH + 5ml Cremaphor}\end{matrix}$$

$$\begin{matrix}\text{FK solution} \left(0.6 \text{mg} / \text{ml}\right) = 1 \text{ml FK stock solution +} \\ \text{19ml phoshpate buffered saline} \left(\text{PBS}\right)\end{matrix}$$

AMD3100

$$\begin{matrix}\text{Stock solution} \left(12 \text{mg} / \text{ml}\right) = 120 \text{mg AMD3100 powder +} \\ \text{10ml H2O} \left(\text{containing 2} \text{.5-3 mg sodium chloride, and adjust to}\right) \\ \text{a pH of 6} \text{.0 to 7} \text{.5 with hydrochloric acid and with sodium} \\ \left(\text{hydroxide, if required}\right)\end{matrix}$$

$$\begin{matrix}\text{AMD3100 solution} \left(\text{1} \text{.2mg} / \text{ml}\right) = 1 \text{ml AMD3100 solution +} \\ \text{9ml PBS}\end{matrix}$$

AF Combination (AMD3100 1 mg/ml; FK506 0.1 mg/ml) = 10 ml AMD3100 solution + 2 ml FK506 solution = 12 mg AMD3100 + 1.2 mg FK506/12 ml = 1 mg AMD3100 + 0.1 mg FK506/ml. The AMD3100/FK506 ratio = 10/1.
Stock solution: Room temperature (aliquoted in 1.0 ml vials)
Dosing for rats and mice: 1 ml/kg
Rat dosing (1ml/kg): volume to be given (ml) = body weight (gram) × 1 ml/1000 gram. For example: a 250 gram rat will be given 0.25 ml subcutaneous injection.

### Composition II (Used for Large Animals Such as Pigs)

FK506

$$\begin{matrix}\text{Stock solution} \left(12 \text{mg} / \text{ml}\right) = \\ 120 \text{mg FK powder + 5ml 95\% EtOH + 5ml Cremaphor}\end{matrix}$$

$$\text{FK solution} \left(6 \text{mg} / \text{ml}\right) = 10 \text{ml FK stock solution + 10ml PBS}$$

AMD3100

$$\begin{matrix}\text{Stock solution} \left(\text{22mg} / \text{ml}\right) = 2200 \text{mg AMD3100 powder +} \\ \text{100ml H2O}\end{matrix}$$

AF combination (AMD3100 1 mg/ml; FK506 0.21 mg/ml) = 90ml AMD3100 stock solution + 10 ml FK506 solution =1980 mg AMD3100 + 60 mgFK506/100 ml. The AMD3100/FK506 ratio = 33/1.

Stock solution: Room temperature (aliquoted in 5 ml vials)

Dosing for pigs: 0.05 ml/kg: volume to be given (ml) = body weight (kg) × 0.05 ml/1 kg. For example: a 50 kg pig will be given 2.5 ml subcutaneous injection.

### Example 3 - Improved Diabetic Wound Healing by AF Combination in Rats

In order to demonstrate the usefulness of the present pharmaceutical compositions and methods to treat diabetic ulcers, the following study was performed with a rat model of diabetes.

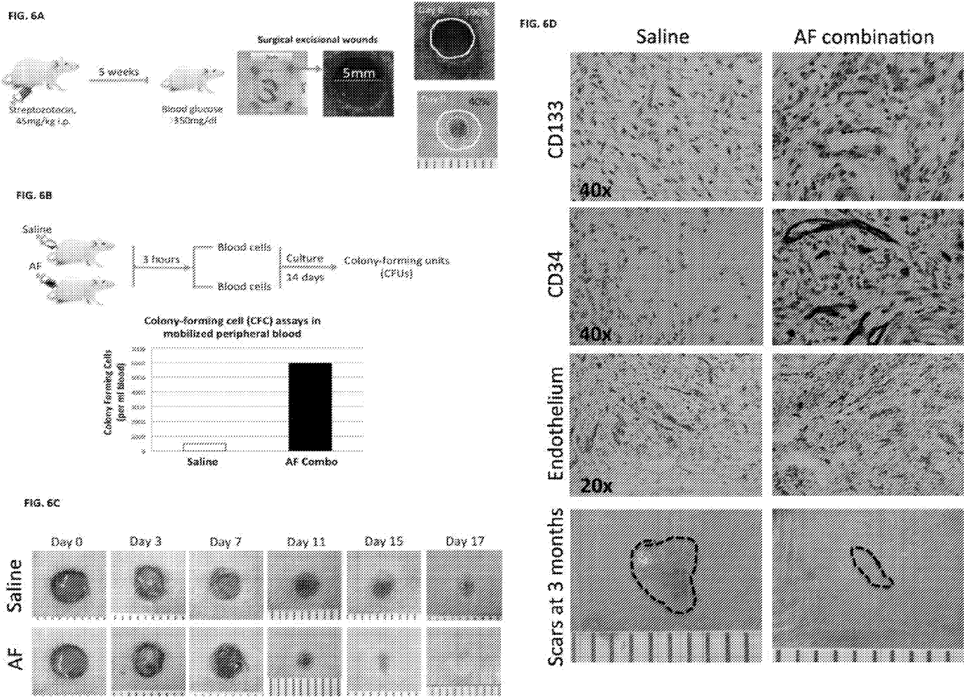

Streptozocin (STZ) induced diabetic SD rats with blood glucose level ≥350 mg/dl for four weeks were used for the study. Full-thickness wounds were created in the dorsal skin of rats with a sterile disposable biopsy punch (5 mm in diameter) (FIG. 6A). Wounded rats were divided randomly into two experimental groups (n=6) and received subcutaneous injections of saline or AF Combination (AMD3100/FK506 ratio = 10/1, AMD3100=1 mg/kg) immediately after wounding and every other day until complete healing was observed. All wound evaluations were double blinded.

The stem cell mobilization activity induced by the AF Combination was measured by using CFC assays in peripheral blood samples at 3 hours after AF Combination treatment, as described above in Example 1. The number of CFC in peripheral blood was increased over 10 times in diabetic rats treated with AF Combination (FIG. 6B), which indicates that bone marrow stem cells can be mobilized by AF combo therapy in diabetic rats. Wounds reached complete closure on day 22 after surgery in diabetic rats treated with saline, which is consistent with the known healing kinetics in this established model. The healing time was reduced to 16 days or by 30% in the diabetic rats treated with AF Combination (FIG. 6C). Healing was accompanied by reduced scar and regeneration of hair follicles. The AF Combination treatment also increased number of CD34+ stem cells and CD133+ endothelial progenitor cells in the wound sites and enhanced capillary and hair follicle neogenesis (FIG. 6D).

In summary, mobilization, recruitment, and retention at wound sites of autochthonous stem cells with AF Combination treatment results in better and faster healing of diabetic wounds, and avoids the isolation, preparation and use of endogenous stem cells which is expensive and time consuming.

### Example 4 - AF Combination Treatment Increases Wound Tensile Strength and Reduces Scarring in an Aged Mouse Model

Impaired wound healing in the elderly presents a major clinical problem. The following study was conducted to show the usefulness of the present compositions and methods for enhancing wound healing in elderly humans by treatment of wounds in an aged mouse model. As discussed below, the AF Combination improved the quality of wound healing and increased wound tensile strength in the aged mice.

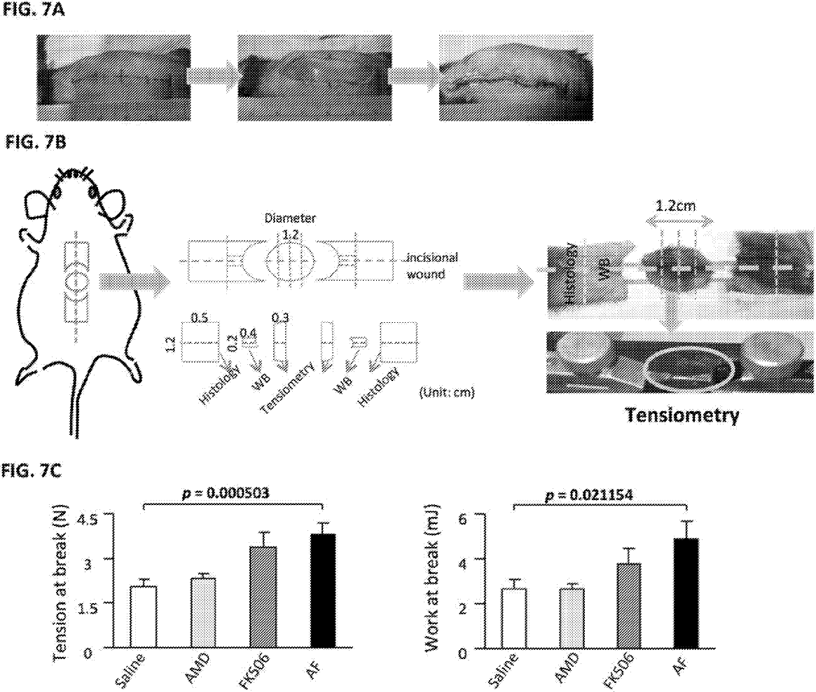

Aged C57BL6 mice underwent incisional wounds (4 cm) (FIG. 7A) and were divided randomly into four experimental groups as follows and received subcutaneous injections of saline or drugs for two weeks after wounding: (1) control-group treated with saline; (2) AMD3100-group treated every other day (1.0 mg kg-1); (3) FK506-group treated daily (0.1 mg kg-1); and (4) a group treated every other day with an AF Combination of AMD3100 and FK506 (AMD3100/FK506 ratio = 10/1). All wound evaluations were double blinded.

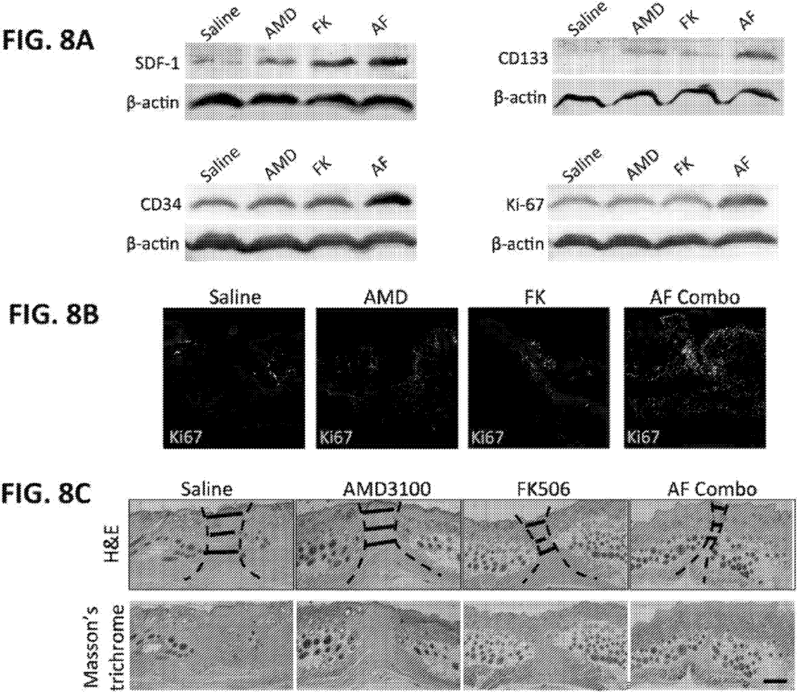

In aged mice, tensile strength of healing wounds was significantly lower than that in younger mice at 21 days post-operation (data not shown). Older mice treated with the AF combination showed significant increases in tensile strength (3.82±0.36 N versus 2.06±0.23 N, p=0.000503) and in the work at break (p=0.021154), restoring the strength of the healing wound to that observed in younger mice (FIG. 7C). Treatment of the aged mice with the AF Combination also increased expression of SDF-1, CD34, CD133 and Ki67 in the wound sites (FIG. 8A), enhanced epithelial proliferation (Ki67+) (FIG. 8B) at 7 days after wounding and reduced scar formation (FIG. 8C). Thus, treatment with the AF Combination resulted in the mobilization, recruitment, and retention at the wound site of endogenous stem cells with AF combination therapy, manifesting in the restoration of healing wound tensile strength and minimizing scars in an aged mouse model of incisional wounds.

### Example 5 - AF Combination Therapy Decreased Colonic Inflammation and Improved Epithelium Regeneration in Mouse Models of Human Inflammatory Bowl Diseases (IBD)

The usefulness of the present composition and methods in treating IBD was demonstrated in mouse models of human IBD.

### Administration of AF Combination Resulted in a Significant Improvement of the Histology and Disease Activity Index in a DSS-Induced Acute Murine Colitis Model

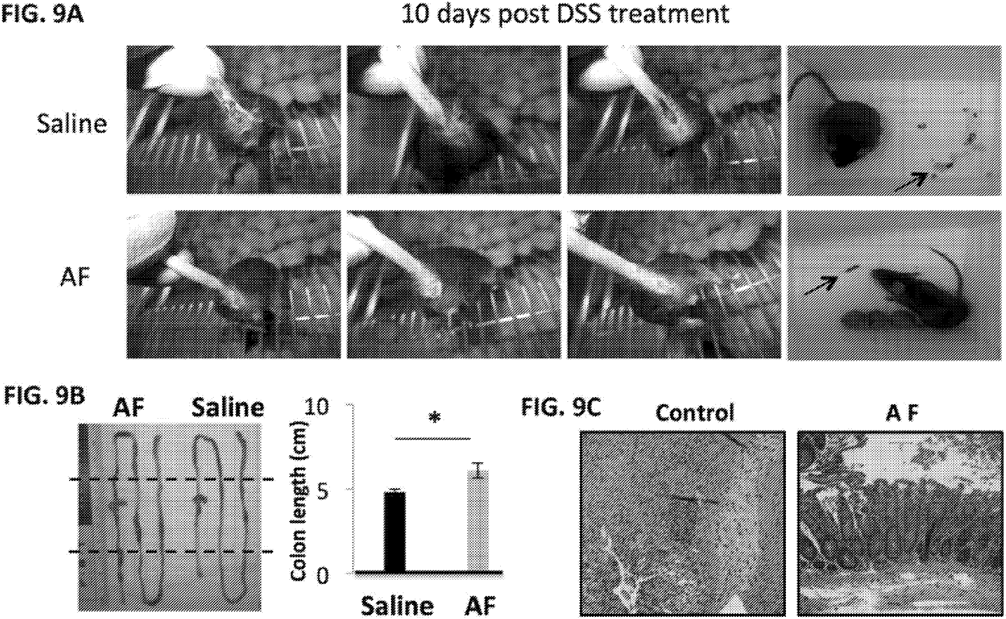

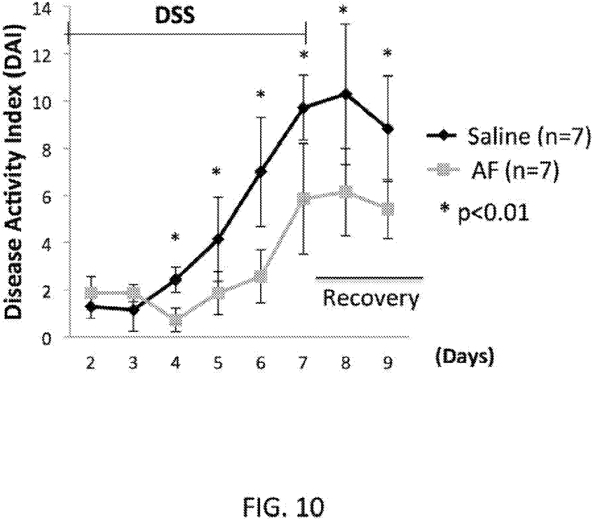

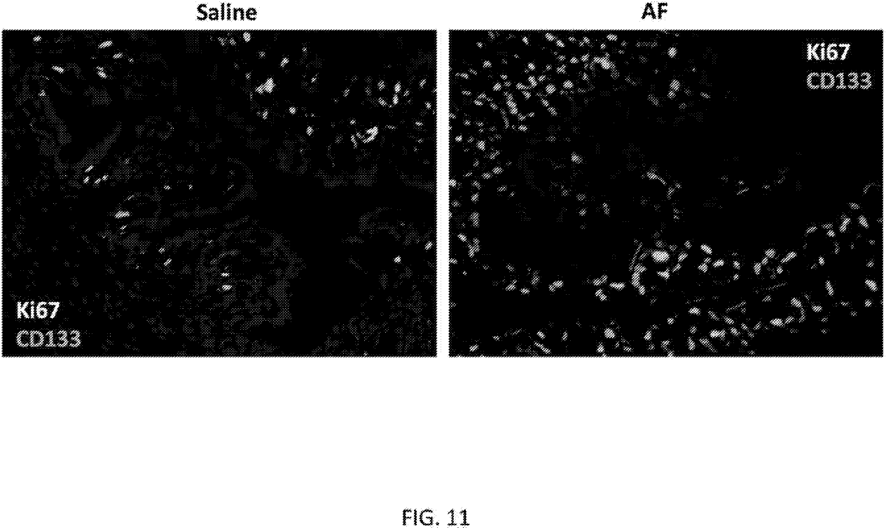

3% dextran sodium sulfate (DSS) (7 days) was used for the induction of DSS-colitis. Mice were divided randomly into two experimental groups and received subcutaneous injections of saline or the AF combination (AMD3100 1 mg/kg and low-dose FK506 0.1 mg/kg, every other day) from day 1 to day 9. Bloody and loose stools (FIG. 9A) and shorter colon and enlarged cecum (FIG. 9B) were observed in saline treated mice compared with the AF combination treated mice at day 10. Histological analysis showed the loss, disruption, or shortening of the crypts and apparent infiltration of inflammatory cells in lamina propria/mucosa in saline treated mice (FIG. 9C). In contrast, no significant intestinal epithelial surface damage or loss of surface epithelial cells was observed in DSS-mice treated with the AF combination. The disease activity index (DAI) in saline-treated mice (controls) progressively increased and peaked at day 8, one day after DSS withdrawal (FIG. 10). However, the DAI were not increased until day 7 in mice treated with the AF Combination. The DAI was almost 50% lower in AF Combination-treated mice compared to that of control mice (FIG. 10). CD133⁺ stem cells and Ki67⁺ proliferating cells were measured in colonic mucosa from saline controls and the AF treated mice by double immunofluorescence staining. A 7-day DSS treatment resulted in damages in the colonic crypt base (FIG. 9C) and reduced the number of CD133⁺ stem cells and Ki67⁺ proliferating cells in the lower portion of the crypt (FIG. 11, left panel; bright staining). However, CD133⁺ stem cells and Ki67⁺ proliferating cells and crypt histology were largely restored in mice with the AF Combination treatment (FIG. 11, right panel; bright staining and FIG. 9C).

### B. AF Combination Therapy Decreased Colonic Inflammation in the IL-10-KO Murine Model of IBD

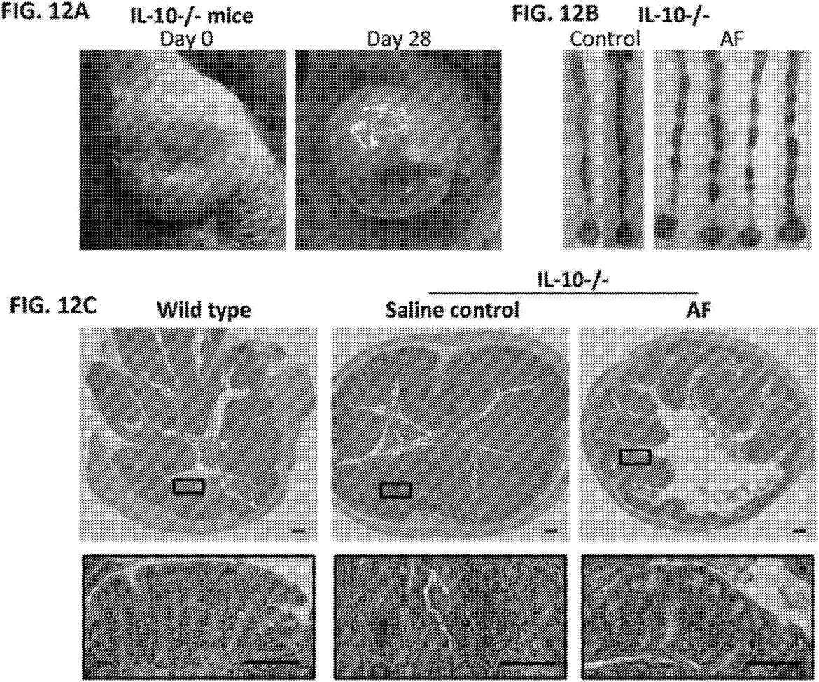

Under the conditions present in the animal facility used, C57BL/6 IL-10KO mice developed spontaneous colitis (IBD) between 3 and 4 months of age. Animals with IBD were treated with the AF Combination (n=7) or saline (control) every other day for 3 weeks and sacrificed 1 week after treatment. Colonic gross and histologic examinations of saline treated mice showed moderate to severe colitis and epithelial hyperplasia with crypt branching (FIGS. 12A and 12B). The AF combination treated mice showed minimal inflammation, similar to wild-type controls (FIG. 12C).

### Example 6 - Improved Recovery After Spinal Cord Injury (SCI) by AF Combination Therapy in Rats

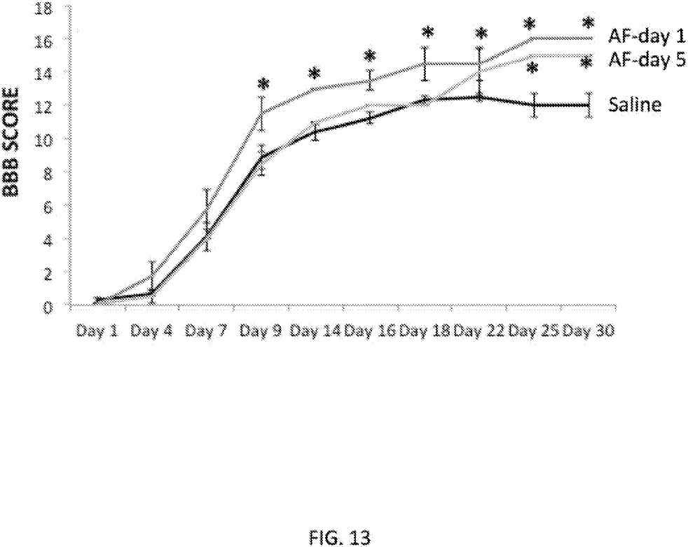

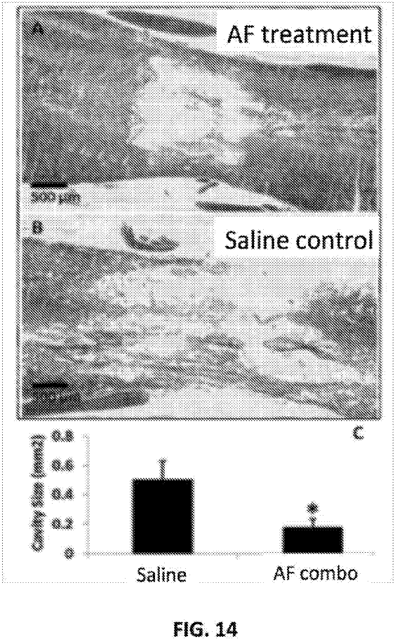

The usefulness of the present compositions and methods for treatment of was demonstrated as follows. A clinically relevant contusive rat model of SCI was generated with weight drops from a height of 12.5 mm to mimic moderate injury. Injured rats were randomly divided into three groups and received subcutaneous injections of saline, the AF Combination (subcutaneous injection, every other day) from day 1 to day 29 or from day 5 to day 29. The average of the Basso, Beattie and Bresnahan Locomotor Rating Scale (BBB score) was used to assess joint movement, hindlimb movements, stepping, limb coordination, trunk position, paw placement and tail position. Statistical differences between BBB score of rat groups were analyzed to assess recovery from the SCI. The data showed that the AF Combination could significantly improve the BBB scale of the contused rats that received treatment 1 and 5 days after SCI (FIG. 13). As expected for a 12.5 mm contusive injury, cavity formation was seen in treatment (FIG. 14A) and control (FIG. 14B) groups; however, the cavitation in spinal cords treated with AF Combination was less well-formed than the control group cavitation. Thus, controls showed more pronounced cavitation, which extended further from the epicenter along the central canal. Area measurements across groups revealed nearly a 3-fold significant reduction in cavity size in the AF Combination treated groups compared to the control group (FIG. 14C), which indicates that treatment with AF Combination limited the normal expected progression of nerve degeneration for this level of contusive injury. These results indicate that pharmacological mobilization of autochthonous stem cells with AF combination promotes spinal cord repair/regeneration and facilities functional recovery in spinal cord injury.

### Example 7 - Induction of In Situ Skin Regeneration by AF Combination Treatment and Skin Allografts in Pigs

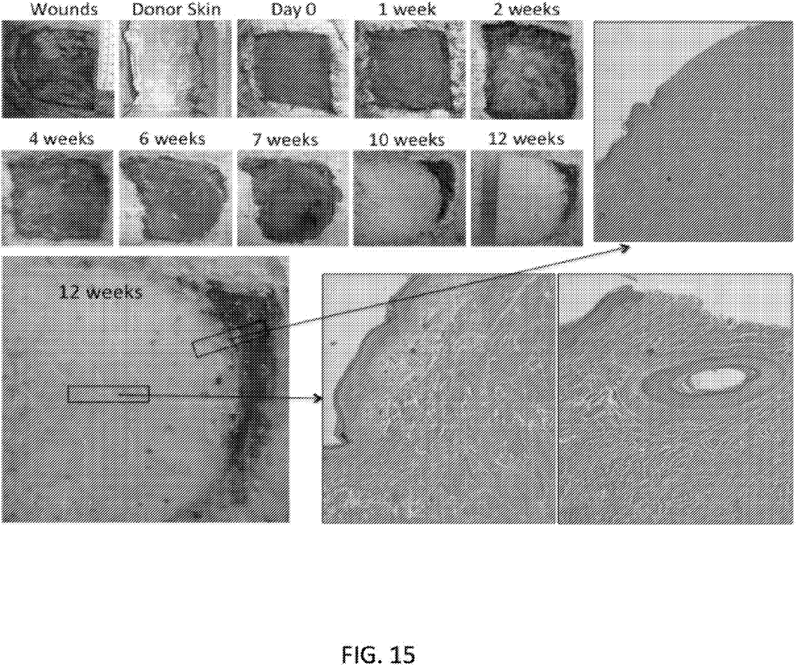

The usefulness of the present pharmaceutical compositions and methods for treating large full thickness burns and soft tissue injuries was demonstrated by the following study. Full-thickness excisional wounds transplanted with split-thickness skin allografts were performed in miniature swine obtained from Transplantation Biology Research Center of Massachusetts General Hospital, Boston, MA. Transplanted swine were treated with AF Combination immediately after skin transplants and every other day for 6 weeks. As shown in FIG. 15, skin allografts were gradually rejected within 4 weeks after transplantation, while wound beds were filled with newly generated tissues. The newly generated tissues were covered by a thin membrane like epithelium with reddish-purple color. The translucent, thin, reddish-purple skin changed to pinkish-red at 7 weeks and became normal skin with hairs at 12 weeks after transplantation. These results indicate that in situ skin regeneration was induced by AF Combination treatment mobilizing and recruiting autochthonous stem cells into the allograft sites, where the skin allografts serve as scaffolds for stem cell regeneration of tissue.

### Example 8 - Long-Term Kidney Transplant Survival of Pigs Treated With AF Combination

The usefulness of the present pharmaceutical compositions and methods for preventing organ transplant rejection, and for promoting long-term survival in organ transplant recipients, was demonstrated with the following study of kidney transplantation performed across full major histocompatibility locus (swine leukocyte antigen [SLA]) mismatches in miniature swine from the Massachusetts General Hospital herd.

Animals - SLA-identified donor and recipient swine (weight 50-80 kg) were obtained from Transplantation Biology Research Center of Massachusetts General Hospital, Boston, MA. The immunogenetic characteristics of these swine have been described previously (30). Two-haplotype full MHC class I and class II mismatched donors and recipients were used for kidney transplantation. All recipients demonstrated a significant in vitro antidonor cytotoxic assay response (>20% specific lysis) before organ transplantation. Institutional review board (Animal Care and Use Committee) approval was obtained for this study. All animal care and procedures were in compliance with the “Principles of Laboratory Animal Care” formulated by the National Society of Medical Research and the “Guide for the Care and Use of Laboratory Animals” prepared by the Institute of Laboratory Animal Resources, National Research Council, and published by the National Academies Press, revised 2011.

Kidney transplantation - The surgical procedure used for kidney transplantation has been described in Kirkman RL, et al. Transplantation in miniature swine. VI. Factors influencing survival of renal allografts. Transplantation 1979; 28: 18-23, the entire disclosure of which is herein incorporated by reference. Briefly, donor kidneys were flushed and persevered in cold saline for 3-6 h before reperfusion. The recipients underwent bilateral nephrectomy. The aorta and inferior vena cava were used for end-to-side arterial and venous anastomoses for the renal artery and vein. The kidney transplantation was completed with a vesicoureteral anastomosis. An indwelling Silastic central venous catheter was placed surgically into the external or internal jugular vein. The catheter facilitated frequent blood sampling for in vitro assays and for monitoring of renal function and whole-blood Tacrolimus levels.

Rejection monitoring - Kidney function was monitored by serial serum creatinine levels. Serum creatinine and blood urea nitrogen levels were analyzed in the Phenotyping Laboratory of the Department of Comparative Medicine at Johns Hopkins. Biopsies were performed on transplant recipients using needle core biopsy via an open or percutaneous ultrasound-guided approach. Renal allograft rejection was defined as sustained rise in serum creatinine to >10 mg/dL or anuria (normal laboratory value for swine serum creatinine is 1-3 mg/dL). Allograft rejection was confirmed histologically in all cases.

Histopathological examination - Core needle biopsies or wedge biopsies were performed on renal allografts. Scoring of acute rejection was based on the Banff classification; see, e.g., Solez K, et al. Banff 07 classification of renal allograft pathology: Updates and future directions. Am J Transplant 2008; 8: 753-760, the entire disclosure of which is herein incorporated by reference. Masson trichrome staining was done according to standard protocols.

SLA-identified and -mismatched inbred miniature swine were subjected to bilateral nephrectomy and transplantation (FIG. 1B) as described in Sachs DH, et al. Transplantation in miniature swine. I. Fixation of the major histocompatibility complex. Transplantation 1976; 22: 559-567, the entire disclosure of which is herein incorporated by reference. Without immunosuppression, these animals die from acute renal failure within 2 weeks, and grafts display histology characteristic of acute cellular rejection (ACR); see, e.g., Kirkman RL, et al. Transplantation in miniature swine. VI. Factors influencing survival of renal allografts. Transplantation 1979; 28: 18-23, supra. Five experimental treatment groups were defined as follows, and each group received subcutaneous injections on days 0, 2, 4, 6, and 8 post-transplantation: (1) saline (n=1); (2) low-dose (0.03 mg/kg) Tacrolimus alone (n=3); (3) AMD3100 1 mg/kg alone (n=3); (4) AF Combination treatment (AMD3100 1 mg/kg and Tacrolimus 0.03 mg/kg) (n=1); and (5) AF Combination treatment as in group 4 but with repeat of the 8-day course at 1, 2, and 3 months post-transplantation.

The results are shown in Table 1. The animal that received no treatment (saline control) developed acute rejection and was euthanized on post-operative day (POD) 10 when the serum creatinine reached 19.4 mg/dL. Two animals in group 2 (n=3) that received low-dose Tacrolimus had prolongation of survival but with markedly elevated serum creatinine, and died from renal failure (anuria for 3 days) on PODs 27 and 28. One animal (21343) that received repeat dosing of Tacrolimus at 1 month reduced the serum creatinine level from 12.3 to 8.3 mg/dL on POD 40 but rebounded to 9.8 mg/dL on POD 56 and became anuric. Group 3 animals (n=3) that received AMD3100 only demonstrated stem cell mobilization but had no prolongation of survival, dying on PODs 9, 10, and 16 with elevated serum creatinine and anuria. In contrast, the group 4 animal that received the perioperative course of the AF Combination treatment survived until POD 240 but was euthanized after elevated creatinine (13.1 mg/dL) and anuria developed. Graft explant histology revealed severe fibrosis but little/mild inflammation. Subsequent miniature swine in the AF Combination treatment group received redosing of the AF Combination at 1, 2, and 3 months (group 5) and one additional 8-day course treatment after skin allograft rejection at 1.5 or 2 years, and these three animals have since received no further medical therapy of any type and remained thriving at 3 years post-transplantation with normal serum creatinine levels (1.8, 2.1, and 2.7 mg/dL) (normal swine creatinine is 1.0-3.0 mg/dL). Notably, swine 21084 had a urinary tract infection that was associated with the development of impaired allograft function (creatinine 22 mg/dL) at 30 days after transplantation. Antibiotics and redosing of the AF Combination improved kidney function, and the serum creatinine was reduced to 3.1 mg/dL at 60 days, 2.5 mg/dL at 100 days, 2.1 mg/dL at 1 year, and 1.8 mg/dL at 3 years post-transplantation.

TABLE 1

Survival of miniature swine after bilateral nephrectomy and mismatched kidney transplantation with stem cell mobilization

| Recipient | Donor | Treatment | ReTreatment | Survival | Creatinine (mg/dL)* |
|---|---|---|---|---|---|
| 21148 CC male | LL female | none | | 10 days | 19.4 |
| 21343 LL female | HH male | Tacro only | Yes | 56 days | 9.8 |
| 21373 GG female | LL male | Tacro only | | 27 days | 9.9 |
| 21370 GG female | LL male | Tacro only | | 28 days | 11.5 |
| 21288 HH female | LL male | AMD3100 only | | 10 days | 22.3 |
| 21146 HH female | LL male | AMD3100 only | | 16 days | 17.3 |
| 21458 GG female | KK male | AMD3100 only | | 9 days | 26.4 |
| 19438 DD female | CC male | AF Combo | No | 240 days | 13.1 |
| 19844 LL female | HH male | AF Combo | Yes | >1200 days | 2.7 |
| 21131 GG female | LL male | AF Combo | Yes | >1170 days | 2.1 |
| 21084 LL female | HH male | AF Combo | Yes | >1020 days | 1.8 |

*Serum creatinine listed for animals at time of death for the first four groups and on days 1215 (animal 19844), 1006 (animal 21131) and 950 (animal 21084).

Comparative survival of all experimental groups is summarized in Table 1. Low-dose FK506 treatment prolonged survival but was ineffective against late rejection. One animal received redosing of FK506 at 30 days but died on POD 56. Animal 19438 received single course of dual-drug treatment reduced serum creatinine levels to 3.1 mg/dL at 1 month but developed late chronic rejection and survived for 240 days. Histology demonstrated inflammation, tubule atrophy, and severe fibrosis. In contrast, animals with repeat dosing at 1, 2, and 3 months survived for the long term with normal kidney function indicating the effectiveness of the AF Combination drug therapy against late/chronic rejection.

### Example 9 - Improved Wound Healing by AF Combination Therapy Versus Separate Administration of AMD3100 and Tacrolimus

The unexpected synergy of AF Combinations was further demonstrated as follows. Male Lewis rats aged 8-12 weeks were anesthetized with isoflurane. The dorsal skin was shaved and cleaned with betadine and 70% ethanol. Four excisional wounds were placed 1 cm to either side of the midline and 1 cm above and below the midpoint between the costal margin and the iliac crests, and were marked by pen. A sterile disposable biopsy punch (5 mm in diameter; Miltex) was aligned vertically over the center of the mark and punched through the skin and panniculus carnosus by applying pressure and twisting at the same time. The same procedure was repeated, generating four wounds on each animal. The animals were house singly after regaining consciousness.

The animals were studied separately and at different times in two experimental groups as follows, and the data were pooled for comparison. 1) The AF combo group (n=6) was treated every other day from wounding (day 0) to complete wound closure with an AF Combination of AMD3100 1 mg/kg and 0.1 mg/kg Tacrolimus, where the ratio of AMD3100 to Tacrolimus = 10/1.2) The A+F group (n=6) was given AMD3100 (1 mg/kg, every other day) and Tacrolimus (0.1 mg/kg, daily) from wounding (day 0) to complete wound closure. The data from both groups was graphed together as the percentage of original wound area over time post-injury to determine dose-responses, which is shown in FIG. 17. The data for the A+F group were originally reported in Lin et al. J Invest Dermatol. 2014 Sep; 134(9): 2458-2468, the entire disclosure of which is herein incorporated by reference.

To obtain the percent wound areas, each wound site on animals from each group was digitally photographed at the indicated time intervals, and wound areas were determined on photographs using Adobe Photoshop (version 7.0; Adobe system). Changes in wound areas over time were expressed as the percentage of the initial wound areas. All wound evaluations were double blinded.

Wounds reached complete closure on day 14 after surgery in the A+F group animals. The 6 animals in the AF combo group exhibited significantly faster healing compared to the A+F group animals, as wounds in the AF combo group reached complete closure at day 13. Thus the AF combo group unexpectedly demonstrated a faster wound healing time while receiving 50% less Tacrolimus compared to animals in the A+F group (0.1 mg/kg every other day for the AF combo group vs. 0.1 mg/kg, daily for the A+F group). The synergistic AF Combination is thus advantageous over the A+F combination, for example in terms of providing faster healing time, administering significantly less dosages of Tacrolimus (which may further avoid undesirable side effects of immunosuppression), and giving less overall injections to the subjects.

1. A method of treating spinal cord injury or demyelination of neurons in a patient comprising administering to the patient a therapeutically effective amount of at least one stem cell mobilizer and at least one immunosuppressive agent.

2. The method of claim 1, wherein the at least one immunosuppressive agent is in the amount of about 0.004 mg/kg to about 0.008 mg/kg.

3. The method of claim 1, wherein the at least one stem cell mobilizer and at least one immunosuppressive agent are administered to the patient via subcutaneous injection, intramuscular injection, oral administration, intravenous injection or intra peritoneal administration.

4. The method of claim 1, wherein the at least one stem cell mobilizer comprises a CXCR4 antagonist.

5. The method of claim 4, wherein the CXCR4 antagonist comprises AMD3100, TG-0054, or AMD3465.

6. The method of claim 5, wherein the CXCR4 antagonist comprises AMD3100.

7. The method of claim 1, wherein the at least one immunosuppressive agent comprises an FK binding protein ligand.

8. The method of claim 1, wherein the at least one immunosuppressive agent comprises Tacrolimus.

9. The method of claim 7, wherein the Tacrolimus is present at about ⅒ the normal dose for immunosuppression.

10. The method of claim 1, wherein the at least one immunosuppressive agent comprises Tacrolimus and wherein the at least one stem cell mobilizer comprises AMD3100.

11. The method of claim 10, where the Tacrolimus and AMD3100 are present in a ratio of about ⅒ to about 1/100.

12. The method of claim 1, wherein the at least one stem cell mobilizer and at least one immunosuppressive agent are administered to the patient via subcutaneous injection or intramuscular injection.

13. The method of claim 12, wherein the at least one stem cell mobilizer and at least one immunosuppressive agent are administered to the patient via subcutaneous injection.

14. The method of claim 1, wherein the at least one stem cell mobilizer and at least one immunosuppressive agent are administered to the patient substantially simultaneously.

15. The method of claim 1, wherein at least one stem cell mobilizer and at least one immunosuppressive agent are administered to the patient every day or every other day.

16. The method of claim 1, wherein at least one stem cell mobilizer and at least one immunosuppressive agent are administered to the patient every other day.

17. A method of treating spinal cord injury or demyelination of neurons in a patient comprising administering to the patient a therapeutically effective amount of (a) a CXCR4 antagonist and (b) an FK binding protein ligand.

18. The method of claim 17, wherein the FK binding protein is in the amount of about 0.004 mg/kg to about 0.008 mg/kg.

19. The method of claim 17, wherein the FK binding protein ligand comprises Tacrolimus or an analog thereof, meridamycin or synthetic ligand of FKBP (SLF).

20. The method of claim 17, wherein the CXCR4 antagonist comprises AMD3100, TG-0054, or AMD3465.
